(19) United States
(12) Patent Application Publication
(10) Pub. No.: US 2002/0138008 A1
(43) Pub. Date: Sep. 26, 2002

(54) Method and apparatus for displaying fluorescence images and method and apparatus for acquiring endoscope images

(76) Inventors: Kazuhiro Tsujita, Kaisei-machi (JP); Toshitaka Agano, Kaisei-machi (JP); Kazuo Hakamata, Kaisei-machi (JP); Katsumi Hayashi, Kaisei-machi (JP); Tomonari Sendai, Kaisei-machi (JP)

Correspondence Address: SUGHRUE, MION, ZINN, MACPEAK & SEAS, PLLC, 2100 Pennsylvania Avenue, N.W.,, Washington, DC, 20037-3202, US

(21) Appl. No.: 09/758,126

(22) Filed: Jan. 12, 2001

(30) Foreign Application Priority Data
Jan. 13, 2000 (JP) 004939/2000
Feb. 18, 2000 (JP) 041777/2000
Sep. 29, 2000 (JP) 299397/2000

(51) Int. Cl. A61B 6/00

(52) U.S. Cl. 600/473; 600/476

(57) ABSTRACT

Operation processing is performed on a first fluorescence image and at least either one of a second fluorescence image and a reflected reference light image to form a tissue condition image, which represents a tissue condition of living body tissues and has been compensated for a distance to the living body tissues. According to at least one of the three images, a judgment is made as to whether each image area in the tissue condition image is an abnormal light affected area or a normal light detection area. The abnormal light affected area is displayed in a form different from the normal light detection area.

### BACKGROUND OF THE INVENTION

1. Field of the Invention

This invention relates to a method and apparatus for displaying a fluorescence image, wherein an image representing at issue condition of living body tissues is displayed in accordance with fluorescence, which has been produced from the living body tissues when excitation light is irradiated to the living body tissues. This invention also relates to a method and apparatus for acquiring an endoscope image, wherein an image of living body tissues is acquired in accordance with reflected light, which has been reflected from the living body tissues when light is irradiated to the living body tissues.

2. Description of the Related Art

There have heretofore been known apparatuses, wherein excitation light is irradiated to living body tissues, intrinsic fluorescence, which has been produced from the living body tissues when the excitation light is irradiated to the living body tissues, is detected as an image, and an image representing a tissue condition of the living body tissues is displayed. For example, there have been proposed endoscope systems, wherein excitation light having a wavelength in the vicinity of 410 nm is irradiated to living body tissues in the body cavity, and an image is formed in accordance with a fluorescence yield or a normalized fluorescence intensity. The fluorescence yield is represented by a ratio of an intensity of fluorescence, which is produced from the living body tissues when the living body tissues are exposed to the excitation light, to an intensity of the excitation light, which is received by the living body tissues. The normalized fluorescence intensity is represented by a ratio of an intensity of fluorescence components of the fluorescence produced from the living body tissues when the living body tissues are exposed to the excitation light, which fluorescence components have wavelengths falling within a wavelength region in the vicinity of 480 nm, to an intensity of fluorescence components of the fluorescence, which fluorescence components have wavelengths falling within a wavelength region of 430 nm to 730 nm. The tissue condition of the living body tissues is seen from the thus formed image.

The fluorescence yield described above is an index utilized for discriminating normal tissues and diseased tissues of a living body from each other in accordance with characteristics such that, in cases where the normal tissues and the diseased tissues receive the excitation light having an identical intensity, the intensity of the intrinsic fluorescence produced from the normal tissues is higher than the intensity of the intrinsic fluorescence produced from the diseased tissues. The thus obtained fluorescence yield is the value represented by the ratio of the intensity of the intrinsic fluorescence, which is produced from a measuring site when the measuring site is exposed to the excitation light, to the intensity of the excitation light, which is received by the same measuring site. Therefore, fluorescence yield is capable of being utilized as a stable index representing the tissue condition of the living body tissues and unaffected by a distance between a radiating-out point, from which the excitation light is radiated out toward the measuring site of the living body tissues, and the measuring site of the living body tissues, which is exposed to the excitation light, an angle of the excitation light with respect to the measuring site, and the like.

In cases where the fluorescence yield is to be calculated, it is not always possible to directly detect the intensity of the excitation light, which is received by the living body tissues. Therefore, actually, the fluorescence yield is calculated by irradiating reference light, such as near infrared light, which has wavelengths falling within a wavelength region such that the light is not apt to be absorbed by the living body tissues, to the living body tissues, detecting an intensity of reflected reference light, which has been reflected from the living body tissues exposed to the reference light, and utilizing the detected intensity of the reflected reference light in lieu of the intensity of the excitation light, which is received by the living body tissues.

Specifically, the fluorescence yield is the value calculated in accordance with the ratio of the intensity of the fluorescence, which has been produced from the living body tissues when the living body tissues are exposed to the excitation light, to the intensity of the excitation light, which is received by the living body tissues. However, as an approximate value of the fluorescence yield, the fluorescence yield is approximately represented by a value calculated in accordance with the ratio of the intensity of the fluorescence, which has been produced from the living body tissues when the living body tissues are exposed to the excitation light, to the intensity of the reflected reference light, which has been reflected from the living body tissues exposed to the reference light.

The normalized fluorescence intensity described above is an index utilized for discriminating the normal tissues and the diseased tissues of the living body from each other in accordance with characteristics such that a spectral pattern of the fluorescence, which is produced from the normal tissues of the living body when the normal tissues are exposed to the excitation light, and the spectral pattern of the fluorescence, which is produced from the diseased tissues of the living body when the diseased tissues are exposed to the excitation light, vary from each other at the wavelength region in the vicinity of 480 nm. As in the cases of the fluorescence yield, the normalized fluorescence intensity is the index unaffected by the distance between the radiating-out point, from which the excitation light is radiated out toward the measuring site of the living body tissues, and the measuring site of the living body tissues, which is exposed to the excitation light, the angle of the excitation light with respect to the measuring site, and the like.

As described above, with the endoscope systems, or the like, with which the tissue condition of the living body tissues in the body cavity is seen as an image, the tissue condition of the living body tissues is seen by use of the tissue condition image, which has been formed by utilizing the index, such as the fluorescence yield or the normalized fluorescence intensity described above.

In cases where the image representing the fluorescence yield is to be formed, when the reference light is irradiated to the living body tissues, it often occurs that the reference light undergoes specular reflection (i.e., regular reflection) from mucus or blood covering the living body tissues, and the reflected light (i.e., the regularly reflected light) passes through a detection optical path and is directly detected. The area on the living body tissues, from which the regularly reflected light has occurred, is detected as a luminous point having a markedly high luminance, which luminous point does not represent the intensity of the excitation light received by the living body tissues. Therefore, an image representing a correct fluorescence yield cannot be obtained from the area described above. Thus a need exists for a technique for eliminating the adverse effects of the regularly reflected light.

As one of ordinary techniques for eliminating the adverse effects of the regularly reflected light, there has heretofore been known a technique, wherein light having been converted by a polarizing filter into linearly polarized light is irradiated to the living body tissues, the light having been reflected from the living body tissues is detected via an optical system, which comprises a polarizing filter located on an imaging side so as to constitute an arrangement of crossed nicols, and the regularly reflected light, in which the direction of polarization of the irradiated light is kept, is thereby removed. As another ordinary technique for eliminating the adverse effects of the regular reflection, there has heretofore been proposed a technique, wherein light having been converted by a polarizing filter into linearly polarized light is irradiated to the living body tissues, and an analyzer is rotated in order to reduce the luminance of the regularly reflected light, which is received, in cases where the luminance of the light, which has been reflected from the living body tissues and has been received by an image sensor, is higher than a predetermined level. As a further ordinary technique for eliminating the adverse effects of the regular reflection, there has heretofore been proposed a technique, wherein a plurality of images containing areas affected by regularly reflected light are detected, corresponding points in the images are detected, and image processing is performed for composing an image such that luminous points due to the regularly reflected light may become imperceptible.

However, in cases where an image representing a tissue condition of living body tissues is to be displayed in accordance with the fluorescence having been produced from the living body tissues, problems should be prevented from occurring in that the image representing the tissue condition of the living body tissues is displayed as an image such that an incorrect diagnosis is made with respect to the tissue condition of the living body tissues. Therefore, in such cases, it is not sufficient that the processing for merely rendering the adverse effects of the regularly reflected light imperceptible is performed in the manner described above.

For example, in cases where the image representing the tissue condition of the living body tissues is to be displayed by the utilization of the fluorescence yield, from the area of the living body tissues, from which the reference light has been regularly reflected, the reflected reference light having a high intensity is detected. Therefore, the aforesaid area is recognized as an area which has received the excitation light having a high intensity. In such cases, the intensity of the fluorescence produced from the aforesaid area and the intensity of the reflected reference light having undergone regular reflection, which reflected reference light has been detected from the aforesaid area, have no relation to each other, and actually the living body tissues at the aforesaid area did not receive the excitation light having a high intensity.

The problems described above cannot be solved sufficiently with the technique described above, wherein the polarizing filter is inserted into the optical path incident upon the image sensor and the intensity of the regularly reflected light is thereby reduced, and the technique described above, wherein the luminous point is rendered imperceptible with the image processing performed in the manner described above. Therefore, due to the adverse effects of the regularly reflected light, an image sufficiently reliable for discriminating the tissue condition of the living body tissues cannot be obtained.

The problems in that an area which cannot accurately express the tissue condition of the living body tissues occurs as described above arises also when the measurement is performed beyond a limit of detection of a measuring device or a limit of an effective measurement range of the measuring device. Also, the problems described above are common to the cases where the fluorescence (the intrinsic fluorescence), which is produced from the living body tissues when the excitation light is irradiated to the living body tissues, is to be detected, and the cases where the fluorescence (extrinsic fluorescence), which is produced from living body tissues having been administered with a fluorescent diagnosis drug when the excitation light is irradiated to the living body tissues, is to be detected.

### SUMMARY OF THE INVENTION

The primary object of the present invention is to provide a method of displaying a fluorescence image, wherein an area embedded in an image representing a tissue condition of living body tissues, at which area the correspondence to the tissue condition of the living body tissues is inaccurate, is manifested, such that the tissue condition of the living body tissues is capable of being seen with a high reliability.

Another object of the present invention is to provide an apparatus for carrying out the method of displaying a fluorescence image.

A further object of the present invention is to provide a method of acquiring an endoscope image, wherein an endoscope image is capable of being acquired such that adverse effects of a luminous point due to regularly reflected light, which luminous point is embedded in an image obtained by detecting reflected light of light having been irradiated to living body tissues and obstructs seeing of the other image areas representing the living body tissues, are reduced.

A still further object of the present invention is to provide an apparatus for carrying out the method of acquiring an endoscope image.

The present invention provides a method of displaying a fluorescence image, wherein operation processing is performed on a first fluorescence image having been obtained by detecting fluorescence components of fluorescence having been produced from living body tissues exposed to excitation light, which fluorescence components have wavelengths falling within a specific wavelength region, and at least either one of a second fluorescence image having been obtained by detecting fluorescence components of the fluorescence, which fluorescence components have wavelengths falling within a wavelength region different from the specific wavelength region, and a reflected reference light image having been obtained by detecting reflected reference light, which has been reflected from the living body tissues when reference light is irradiated to the living body tissues, a tissue condition image, which represents a tissue condition of the living body tissues and which has been compensated for a distance to the living body tissues, is formed with the operation processing, and the thus formed tissue condition image is displayed, the method comprising the steps of:

i) making a judgment as to whether each of image areas embedded in the tissue condition image is an abnormal light affected area, which has been affected by light having an intensity equal to at least a specified value, or a normal light detection area, which has been formed with light having an intensity lower than the specified value, the judgment being made in accordance with at least one image, which is among the first fluorescence image, the second fluorescence image, and the reflected reference light image, and

ii) displaying the abnormal light affected area in a form different from the normal light detection area.

The present invention also provides an apparatus for displaying a fluorescence image, wherein operation processing is performed on a first fluorescence image having been obtained by detecting fluorescence components of fluorescence having been produced from living body tissues exposed to excitation light, which fluorescence components have wavelengths falling within a specific wavelength region, and at least either one of a second fluorescence image having been obtained by detecting fluorescence components of the fluorescence, which fluorescence components have wavelengths falling within a wavelength region different from the specific wavelength region, and a reflected reference light image having been obtained by detecting reflected reference light, which has been reflected from the living body tissues when reference light is irradiated to the living body tissues, a tissue condition image, which represents a tissue condition of the living body tissues and which has been compensated for a distance to the living body tissues, is formed with the operation processing, and the thus formed tissue condition image is displayed, the apparatus comprising:

i) judgment means for making a judgment as to whether each of image areas embedded in the tissue condition image is an abnormal light affected area, which has been affected by light having an intensity equal to at least a specified value, or a normal light detection area, which has been formed with light having an intensity lower than the specified value, the judgment being made in accordance with at least one image, which is among the first fluorescence image, the second fluorescence image, and the reflected reference light image, and

ii) abnormal light affected area displaying means for receiving an output from the judgment means and displaying the abnormal light affected area in a form different from the normal light detection area in accordance with the output received from the judgment means.

Specifically, the method and apparatus for displaying a fluorescence image in accordance with the present invention is characterized by displaying the abnormal light affected area, which has been affected by light having an intensity equal to at least the specified value and which is not reliable, in a form such that the abnormal light affected area is capable of being discriminated from the normal light detection area.

In the method and apparatus for displaying a fluorescence image in accordance with the present invention, the specified value should preferably be determined in accordance with an intensity of the reflected reference light, which intensity indicates the presence of regularly reflected light, in the reflected reference light image. Alternatively, the specified value should preferably be determined in accordance with a limit of the detection in at least one image, which is among the first fluorescence image, the second fluorescence image, and the reflected reference light image. As another alternative, the specified value should preferably be determined in accordance with a limit of an effective measurement range in at least one image, which is among the first fluorescence image, the second fluorescence image, and the reflected reference light image.

Also, in the apparatus for displaying a fluorescence image in accordance with the present invention, the abnormal light affected area displaying means may display the abnormal light affected area in the form different from the normal light detection area only in cases where the tissue condition image is displayed as a still image.

Further, in the apparatus for displaying a fluorescence image in accordance with the present invention, the tissue condition image should preferably represent a fluorescence yield or a normalized fluorescence intensity.

Furthermore, the apparatus for displaying a fluorescence image in accordance with the present invention may be modified such that at least one image, which is among the first fluorescence image, the second fluorescence image, and the reflected reference light image, is obtained from photoelectric detection of light with an image sensor, and the limit of the detection corresponds to a saturation value of an output of the image sensor.

Also, the apparatus for displaying a fluorescence image in accordance with the present invention should preferably be modified such that a calculation is made to find a mean value of detected values of at least either one of the first fluorescence image and the second fluorescence image, which have been obtained by detecting the fluorescence having been produced from normal tissues when the excitation light is irradiated to the normal tissues spaced apart by a predetermined distance from an excitation light radiating-out point, and the specified value in accordance with the limit of the effective measurement range is determined in accordance with a value, which is obtained by adding a value representing a variation of the detected values to the thus calculated mean value.

Further, the apparatus for displaying a fluorescence image in accordance with the present invention may be modified such that the abnormal light affected area displaying means displays the abnormal light affected area as a color area in cases where the normal light detection area is displayed as a monochromatic area, and the abnormal light affected area displaying means displays the abnormal light affected area as a monochromatic area in cases where the normal light detection area is displayed as a color area.

Alternatively, the abnormal light affected area displaying means displays the abnormal light affected area as a blinking area.

Furthermore, the apparatus for displaying a fluorescence image in accordance with the present invention may further comprise displaying change-over means for manually changing over between an abnormal light affected area displaying mode and an abnormal light affected area non-displaying mode.

Also, the apparatus for displaying a fluorescence image in accordance with the present invention may be constituted as an endoscope system provided with an endoscope tube to be inserted into a living body.

Further, the apparatus for displaying a fluorescence image in accordance with the present invention may be modified such that the apparatus further comprises a light source for producing the excitation light, and the light source is a GaN type of semiconductor laser. The wavelength of a laser beam produced by the GaN type of semiconductor laser should preferably fall within the range of 400 nm to 420 nm.

The term “effective measurement range” as used herein means the range determined in accordance with the performance of the optical system which the apparatus for displaying a fluorescence image has, or the like. For example, the term “effective measurement range” as used herein means the range, over which the living body tissues are capable of being seen accurately and which is determined by a depth of field of the optical system.

Also, the term “predetermined distance” as used herein means the distance at the time at which the excitation light radiating-out point is closest to the living body tissues within the effective measurement range.

Further, the term “form” as used herein means, for example, a color, a shape, a pattern, and the presence or absence of blinking.

Furthermore, the term “each of image areas embedded in a tissue condition image” as used herein means the area of a pixel in the tissue condition image, the area of a group of multiple pixels in the tissue condition image, or the like.

The fluorescence yield need not necessarily be the value calculated in accordance with the ratio of the intensity of the fluorescence, which has been produced from the living body tissues when the living body tissues are exposed to the excitation light, to the intensity of the excitation light, which is received by the living body tissues. For example, the fluorescence yield may be a value, which has been calculated approximately by use of substitute light, or the like. The value having been calculated approximately is herein also referred to as the fluorescence yield.

The present invention further provides a first method of acquiring an endoscope image, comprising the steps of:

i) irradiating light to living body tissues,

ii) detecting reflected light, which has been reflected from the living body tissues when the light is irradiated to the living body tissues, as an image, and

iii) acquiring a reflection image from the image, which has been obtained by detecting the reflected light,

wherein the reflection image is acquired by performing low-pass filtering processing on the image, which has been obtained from the detection of the reflected light.

The present invention still further provides a second method of acquiring an endoscope image, comprising the steps of:

i) irradiating light to living body tissues,

ii) detecting reflected light, which has been reflected from the living body tissues when the light is irradiated to the living body tissues, as an image, and

iii) acquiring a reflection image from the image, which has been obtained by detecting the reflected light,

wherein the reflection image is acquired by:

performing differentiation filtering processing on the image, which has been obtained from the detection of the reflected light, in order to specify a regular reflection image area, which is embedded in the image having been obtained from the detection of the reflected light and is affected by regularly reflected light of the light having been irradiated to the living body tissues, and

substituting an image value within the regular reflection image area by a corrected value, which is determined in accordance with image values at an area surrounding the regular reflection image area.

The present invention also provides a third method of acquiring an endoscope image, comprising the steps of:

i) irradiating light to living body tissues,

ii) detecting reflected light, which has been reflected from the living body tissues when the light is irradiated to the living body tissues, as an image, and

iii) acquiring a reflection image from the image, which has been obtained by detecting the reflected light,

wherein the irradiation of the light is performed from two different positions and with different timings,

the reflected light, which has been reflected from the living body tissues when the light is irradiated from one of the two different positions to the living body tissues, and the reflected light, which has been reflected from the living body tissues when the light is irradiated from the other position to the living body tissues, are detected respectively as two images, and

the reflection image is acquired by:

calculating a difference between the two detected images in order to specify regular reflection image areas, which are embedded respectively in the two detected images and are affected by regularly reflected light of the light having been irradiated to the living body tissues,

substituting an image value within each of the regular reflection image areas, which are embedded respectively in the two detected images, by a corrected value, which is determined in accordance with image values at an area surrounding the corresponding regular reflection image area, and

adding two images, which have been obtained from the substitution, to each other.

The present invention further provides a fourth method of acquiring an endoscope image, comprising the steps of:

i) irradiating light to living body tissues,

ii) detecting reflected light, which has been reflected from the living body tissues when the light is irradiated to the living body tissues, as an image, and

iii) acquiring a reflection image from the image, which has been obtained by detecting the reflected light,

wherein the irradiation of the light is performed from two different positions and with different timings,

the reflected light, which has been reflected from the living body tissues when the light is irradiated from one of the two different positions to the living body tissues, and the reflected light, which has been reflected from the living body tissues when the light is irradiated from the other position to the living body tissues, are detected respectively as two images, and

the reflection image is acquired by:

performing low-pass filtering processing on each of the two detected images, and

adding two images, which have been obtained from the low-pass filtering processing, to each other.

The present invention still further provides a first apparatus for acquiring an endoscope image, comprising:

i) irradiation means for irradiating light to living body tissues,

ii) detection means for detecting reflected light, which has been reflected from the living body tissues when the light is irradiated to the living body tissues, as an image, and

iii) image acquiring means for acquiring a reflection image from the image, which has been obtained by detecting the reflected light,

wherein the image acquiring means acquires the reflection image by performing low-pass filtering processing on the image, which has been obtained from the detection of the reflected light.

In the first apparatus for acquiring an endoscope image in accordance with the present invention, the low-pass filtering processing may be one-dimensional low-pass filtering processing.

Alternatively, in the first apparatus for acquiring an endoscope image in accordance with the present invention, the low-pass filtering processing may be two-dimensional low-pass filtering processing.

The present invention also provides a second apparatus for acquiring an endoscope image, comprising:

i) irradiation means for irradiating light to living body tissues,

ii) detection means for detecting reflected light, which has been reflected from the living body tissues when the light is irradiated to the living body tissues, as an image, and

iii) image acquiring means for acquiring a reflection image from the image, which has been obtained by detecting the reflected light,

wherein the image acquiring means acquires the reflection image by:

performing differentiation filtering processing on the image, which has been obtained from the detection of the reflected light, in order to specify a regular reflection image area, which is embedded in the image having been obtained from the detection of the reflected light and is affected by regularly reflected light of the light having been irradiated to the living body tissues, and

substituting an image value within the regular reflection image area by a corrected value, which is determined in accordance with image values at an area surrounding the regular reflection image area.

In the second apparatus for acquiring an endoscope image in accordance with the present invention, the differentiation filtering processing may be one-dimensional differentiation filtering processing.

Alternatively, in the second apparatus for acquiring an endoscope image in accordance with the present invention, the differentiation filtering processing may be two-dimensional differentiation filtering processing.

The present invention further provides a third apparatus for acquiring an endoscope image, comprising:

i) irradiation means for irradiating light to living body tissues,

ii) detection means for detecting reflected light, which has been reflected from the living body tissues when the light is irradiated to the living body tissues, as an image, and

iii) image acquiring means for acquiring a reflection image from the image, which has been obtained by detecting the reflected light,

wherein the irradiation means irradiates the light from two different positions and with different timings to the living body tissues,

the detection means detects the reflected light, which has been reflected from the living body tissues when the light is irradiated from one of the two different positions to the living body tissues, and the reflected light, which has been reflected from the living body tissues when the light is irradiated from the other position to the living body tissues, respectively as two images, and

the image acquiring means acquires the reflection image by:

calculating a difference between the two detected images in order to specify regular reflection image areas, which are embedded respectively in the two detected images and are affected by regularly reflected light of the light having been irradiated to the living body tissues,

substituting an image value within each of the regular reflection image areas, which are embedded respectively in the two detected images, by a corrected value, which is determined in accordance with image values at an area surrounding the corresponding regular reflection image area, and

adding two images, which have been obtained from the substitution, to each other.

The present invention further provides a fourth apparatus for acquiring an endoscope image, comprising:

i) irradiation means for irradiating light to living body tissues,

ii) detection means for detecting reflected light, which has been reflected from the living body tissues when the light is irradiated to the living body tissues, as an image, and

iii) image acquiring means for acquiring a reflection image from the image, which has been obtained by detecting the reflected light,

wherein the irradiation means irradiates the light from two different positions and with different timings to the living body tissues,

the detection means detects the reflected light, which has been reflected from the living body tissues when the light is irradiated from one of the two different positions to the living body tissues, and the reflected light, which has been reflected from the living body tissues when the light is irradiated from the other position to the living body tissues, respectively as two images, and

the image acquiring means acquires the reflection image by:

performing low-pass filtering processing on each of the two detected images, and

adding two images, which have been obtained from the low-pass filtering processing, to each other.

Each of the first, second, third, and fourth apparatuses for acquiring an endoscope image in accordance with the present invention may be modified such that the apparatus further comprises excitation light irradiating means for irradiating excitation light to the living body tissues, the excitation light causing the living body tissues to produce fluorescence, and fluorescence image detecting means for detecting the fluorescence, which has been produced from the living body tissues when the excitation light is irradiated to the living body tissues, as a fluorescence image, and

the image acquiring means acquires a fluorescence yield image in accordance with a ratio of the fluorescence image to the reflection image.

In such cases, the reflection image may be an image formed with reflected light of the excitation light.

Alternatively, in such cases, the reflection image may be an image formed with reflected light of near infrared light, which has been irradiated by the irradiation means to the living body tissues.

As another alternative, in such cases, the reflection image may be an image formed with reflected light of light, which has wavelengths falling within a red wavelength region and has been irradiated by the irradiation means to the living body tissues.

As a further alternative, in such cases, the reflection image may be an image formed with a luminance signal having been formed in accordance with the reflected light of the light, which has been irradiated by the irradiation means to the living body tissues.

The term “luminance signal” as used herein means the signal representing the luminance of the image, which is obtained by combining R, G, and B three primary color signals in video signals.

With the method and apparatus for displaying a fluorescence image in accordance with the present invention, wherein the operation processing is performed on the first fluorescence image and at least either one of the second fluorescence image and the reflected reference light image, and the tissue condition image representing the tissue condition of the living body tissues is formed from the operation processing and displayed, the abnormal light affected area, which has been affected by light having an intensity equal to at least the specified value, is displayed in a form different from the normal light detection area, which has been formed with light having an intensity lower than the specified value. Therefore, the abnormal light affected area, which has been affected by light having an intensity equal to at least the specified value and at which the correspondence to the tissue condition of the living body tissues is inaccurate, and the normal light detection area, at which the correspondence to the tissue condition of the living body tissues is accurate, are capable of being easily discriminated from each other. Accordingly, only the normal light detection area is capable of being taken as an area to be seen. As a result, the tissue condition of the living body tissues is capable of being seen with a high reliability.

Also, with the method and apparatus for displaying a fluorescence image in accordance with the present invention, the specified value maybe determined in accordance with the intensity of the reflected reference light, which intensity indicates the presence of the regularly reflected light, in the reflected reference light image. Alternatively, the specified value may be determined in accordance with the limit of the detection in at least one image, which is among the first fluorescence image, the second fluorescence image, and the reflected reference light image. As another alternative, the specified value may be determined in accordance with the limit of the effective measurement range in at least one image, which is among the first fluorescence image, the second fluorescence image, and the reflected reference light image. In such cases, the abnormal light affected area is capable of being determined more accurately.

Further, in the apparatus for displaying a fluorescence image in accordance with the present invention, the abnormal light affected area displaying means may display the abnormal light affected area in the form different from the normal light detection area only in cases where the tissue condition image is displayed as a still image. In such cases, for example, when a site in the living body, which site is to be seen, is being searched, the abnormal light affected area may not be displayed, and the tissue condition image may be displayed as a dynamic image. Also, after the site to be seen has been searched, the tissue condition image may be displayed as a still image such that the details of the tissue condition maybe seen. Only when the tissue condition image is thus displayed as a still image, the abnormal light affected area may be displayed. Specifically, when the person, who sees the displayed image, is searching the site to be seen and is not paying attention to the tissue condition of the living body tissues, the abnormal light affected area does not come into the visual field. Therefore, the burden to the person, who sees the displayed image, is capable of being kept light. Also, when the site to be seen is being searched, the abnormal light affected area need not be displayed in the real time mode (as the dynamic image) through quick operation processing. Therefore, the burden to devices, such as a microprocessor and a memory, is capable of being kept light.

Furthermore, with the apparatus for displaying a fluorescence image in accordance with the present invention, wherein the tissue condition image represents the fluorescence yield or the normalized fluorescence intensity, the tissue condition is capable of being seen more reliably. Specifically, it has been known that the fluorescence yield and the normalized fluorescence intensity are the values reflecting the tissue condition of the living body tissues. Therefore, in cases where the tissue condition image is approximately represented by the fluorescence yield or the normalized fluorescence intensity, the tissue condition of the living body tissues is capable of being seen more reliably.

Also, with the apparatus for displaying a fluorescence image in accordance with the present invention, at least one image, which is among the first fluorescence image, the second fluorescence image, and the reflected reference light image, may be obtained from photoelectric detection of light with an image sensor, and the limit of the detection may correspond to the saturation value of the output of the image sensor. In such cases, the specified value becomes clear, and the abnormal light affected area is capable of being determined more accurately.

Further, with the apparatus for displaying a fluorescence image in accordance with the present invention, a calculation may be made to find the mean value of the detected values of at least either one of the first fluorescence image and the second fluorescence image, which have been obtained by detecting the fluorescence having been produced from the normal tissues when the excitation light is irradiated to the normal tissues spaced apart by the predetermined distance from the excitation light radiating-outpoint. Also, the specified value in accordance with the limit of the effective measurement range may be determined in accordance with the value, which is obtained by adding the value representing the variation of the detected values to the thus calculated mean value. In such cases, the specified value of the effective measurement range is capable of being calculated statistically, and the abnormal light affected area is capable of being determined more accurately.

Furthermore, with the apparatus for displaying a fluorescence image in accordance with the present invention, the abnormal light affected area displaying means may display the abnormal light affected area as a color area in cases where the normal light detection area is displayed as a monochromatic area. Also, the abnormal light affected area displaying means may display the abnormal light affected area as a monochromatic area in cases where the normal light detection area is displayed as a color area. Alternatively, the abnormal light affected area displaying means may display the abnormal light affected area as a blinking area. In such cases, the abnormal light affected area is capable of being discriminated more reliably.

Also, with the apparatus for displaying a fluorescence image in accordance with the present invention, wherein the apparatus further comprises the displaying change-over means for manually changing over between the abnormal light affected area displaying mode and the abnormal light affected area non-displaying mode, the tissue condition of the living body tissues is capable of being displayed such that the person, who sees the displayed image, is capable of easily seeing the tissue condition of the living body tissues.

Further, with the apparatus for displaying a fluorescence image in accordance with the present invention, which is constituted as the endoscope system provided with the endoscope tube to be inserted into a living body, a region inside of the living body is capable of being seen more easily.

Furthermore, with the apparatus for displaying a fluorescence image in accordance with the present invention, wherein the light source for producing the excitation light is the GaN type of semiconductor laser, the apparatus is capable of being kept small in size and cheap in cost.

With the first method and apparatus for acquiring an endoscope image in accordance with the present invention, the reflected light, which has been reflected from the living body tissues when the light is irradiated to the living body tissues, is detected as an image, and the reflection image is acquired from the image, which has been obtained by detecting the reflected light. In such cases, the reflection image is acquired by performing the low-pass filtering processing (such as one-dimensional low-pass filtering processing or two-dimensional low-pass filtering processing) on the image, which has been obtained from the detection of the reflected light. Therefore, the degree of change in image value of the regular reflection image area, which exhibits a sharp change in luminance and is affected by the regularly reflected light contained in the reflected light having been detected, is capable of being suppressed. As a result, the reflection image is capable of being acquired such that adverse effects of a luminous point due to the regularly reflected light, which luminous point is embedded in the image obtained by detecting the reflected light of the light having been irradiated to the living body tissues and obstructs the seeing of the other image areas representing the living body tissues, are reduced.

With the second method and apparatus for acquiring an endoscope image in accordance with the present invention, the reflected light, which has been reflected from the living body tissues when the light is irradiated to the living body tissues, is detected as an image, and the reflection image is acquired from the image, which has been obtained by detecting the reflected light. In such cases, the differentiation filtering processing (such as one-dimensional differentiation filtering processing or two-dimensional differentiation filtering processing) is performed on the image, which has been obtained from the detection of the reflected light, in order to specify the regular reflection image area, which is embedded in the image having been obtained from the detection of the reflected light and is affected by regularly reflected light of the light having been irradiated to the living body tissues. Also, the image value within the regular reflection image area is substituted by the corrected value, which is determined in accordance with the image values at the area surrounding the regular reflection image area. Therefore, the image area affected by the regularly reflected light, which is contained in the reflected light having been detected and has a markedly high luminance, i.e. the regular reflection image area, is capable of being specified accurately. Also, the image value in the image area representing the high luminance of the regularly reflected light is substituted so as to become approximately identical with the image values of the surrounding area, which image values are appropriate for the seeing of the image pattern of the living body tissues. As a result, the reflection image is capable of being acquired such that adverse effects of a luminous point due to the regularly reflected light, which luminous point is embedded in the image obtained by detecting the reflected light of the light having been irradiated to the living body tissues and obstructs the seeing of the other image areas representing the living body tissues, are reduced.

With the third method and apparatus for acquiring an endoscope image in accordance with the present invention, the reflected light, which has been reflected from the living body tissues when the light is irradiated to the living body tissues, is detected as an image, and the reflection image is acquired from the image, which has been obtained by detecting the reflected light. In such cases, the irradiation of the light is performed from the two different positions and with the different timings. Also, the reflected light, which has been reflected from the living body tissues when the light is irradiated from one of the two different positions to the living body tissues, and the reflected light, which has been reflected from the living body tissues when the light is irradiated from the other position to the living body tissues, are detected respectively as the two images. Further, the difference between the two detected images is calculated in order to specify the regular reflection image areas, which are embedded respectively in the two detected images and are affected by the regularly reflected light of the light having been irradiated to the living body tissues. Furthermore, the image value within each of the regular reflection image areas, which are embedded respectively in the two detected images, is substituted by the corrected value, which is determined in accordance with the image values at the area surrounding the corresponding regular reflection image area. Thereafter, the two images, which have been obtained from the substitution, are added to each other. Therefore, the image areas affected by the regularly reflected light, i.e., the regular reflection image areas, which occur at different positions in the two detected images due to the difference in position of radiating-out of the light, are capable of being specified accurately. Also, in each of the two detected images, the image value in the image area representing the high luminance of the regularly reflected light is substituted so as to become approximately identical with the image values of the surrounding area, which image values are appropriate for the seeing of the image pattern of the living body tissues. As a result, the reflection image is capable of being acquired such that adverse effects of a luminous point due to the regularly reflected light, which luminous point is embedded in the image obtained by detecting the reflected light of the light having been irradiated to the living body tissues and obstructs the seeing of the other image areas representing the living body tissues, are reduced.

With the fourth method and apparatus for acquiring an endoscope image in accordance with the present invention, the reflected light, which has been reflected from the living body tissues when the light is irradiated to the living body tissues, is detected as an image, and the reflection image is acquired from the image, which has been obtained by detecting the reflected light. In such cases, the irradiation of the light is performed from the two different positions and with the different timings. Also, the reflected light, which has been reflected from the living body tissues when the light is irradiated from one of the two different positions to the living body tissues, and the reflected light, which has been reflected from the living body tissues when the light is irradiated from the other position to the living body tissues, are detected respectively as the two images. Further, the low-pass filtering processing is performed on each of the two detected images, and the two images, which have been obtained from the low-pass filtering processing, are added to each other. Therefore, the image areas affected by the regularly reflected light, i.e., the regular reflection image areas, which occur at different positions in the two detected images due to the difference in position of radiating-out of the light, are capable of being specified accurately. Also, in each of the two detected images, the degree of change in image value of the regular reflection image area, which exhibits a sharp change in luminance and is affected by the regularly reflected light contained in the reflected light having been detected, is capable of being suppressed. As a result, the reflection image is capable of being acquired such that adverse effects of a luminous point due to the regularly reflected light, which luminous point is embedded in the image obtained by detecting the reflected light of the light having been irradiated to the living body tissues and obstructs the seeing of the other image areas representing the living body tissues, are reduced.

Each of the first, second, third, and fourth apparatuses for acquiring an endoscope image in accordance with the present invention may be modified such that the apparatus further comprises the excitation light irradiating means for irradiating the excitation light to the living body tissues, the excitation light causing the living body tissues to produce the fluorescence, and the fluorescence image detecting means for detecting the fluorescence, which has been produced from the living body tissues when the excitation light is irradiated to the living body tissues, as the fluorescence image, and such that the image acquiring means acquires the fluorescence yield image in accordance with the ratio of the fluorescence image to the reflection image. With the modification, the fluorescence yield image, which accurately represents the fluorescence yield, is capable of being acquired.

With the modification of each of the first, second, third, and fourth apparatuses for acquiring an endoscope image in accordance with the present invention, wherein the reflection image is the image formed with the reflected light of the excitation light, the fluorescence yield image, which accurately represents the fluorescence yield, is capable of being acquired.

With the modification of each of the first, second, third, and fourth apparatuses for acquiring an endoscope image in accordance with the present invention, wherein the reflection image is the image formed with the reflected light of the near infrared light, which has been irradiated by the irradiation means to the living body tissues, the fluorescence yield image, which accurately represents the fluorescence yield, is capable of being acquired.

With the modification of each of the first, second, third, and fourth apparatuses for acquiring an endoscope image in accordance with the present invention, wherein the reflection image is the image formed with the reflected light of the light, which has wavelengths falling within the red wavelength region and has been irradiated by the irradiation means to the living body tissues, the fluorescence yield image, which accurately represents the fluorescence yield, is capable of being acquired.

With the modification of each of the first, second, third, and fourth apparatuses for acquiring an endoscope image in accordance with the present invention, wherein the reflection image is the image formed with the luminance signal having been formed in accordance with the reflected light of the light, which has been irradiated by the irradiation means to the living body tissues, the fluorescence yield image, which accurately represents the fluorescence yield, is capable of being acquired.

### BRIEF DESCRIPTION OF THE DRAWINGS

FIG. 1 is a schematic view showing a fluorescence endoscope system, in which a first embodiment of the apparatus for displaying a fluorescence image in accordance with the present invention is employed,

FIG. 2 is an explanatory view showing a rotating filter,

FIG. 3 is a timing chart showing timings, with which light beams having wavelengths falling within different wavelength regions are irradiated,

FIG. 4 is an explanatory view showing how a regularly reflected light area is recognized by the utilization of a threshold value Q,

FIG. 5A is an explanatory view showing a reflected reference light image Zn,

FIG. 5B is an explanatory view showing a fluorescence image Zk,

FIG. 5C is an explanatory view showing a surface sequential light image Zm,

FIG. 6 is an explanatory view showing how the reference light image Zn, the fluorescence image Zk, and the surface sequential light image Zm are superimposed one upon another,

FIG. 7 is an explanatory view showing a tissue condition image, which is displayed,

FIG. 8 is a schematic view showing a different example of how the fluorescence image Zk, and the like, are detected,

FIG. 9 is a schematic view showing a fluorescence endoscope system, in which a second embodiment of the apparatus for displaying a fluorescence image in accordance with the present invention is employed,

FIG. 10 is an explanatory view showing a rotating filter,

FIG. 11 is an explanatory view showing how an abnormal light affected area is determined in accordance with a logical product of transfinite areas embedded in images,

FIG. 12 is an explanatory view showing how a composed image is formed such that an abnormal light affected area is displayed in a tissue condition image,

FIG. 13 is a block diagram showing displaying change-over means for manually changing over between an abnormal light affected area displaying mode and an abnormal light affected area non-displaying mode,

FIG. 14 is a schematic view showing a fluorescence endoscope system, in which a first embodiment of the apparatus for acquiring an endoscope image in accordance with the present invention is employed,

FIG. 15 is an explanatory view showing a rotating filter employed in the fluorescence endoscope system of FIG. 14,

FIG. 16 is an explanatory view showing a rotating reflection-transmission plate,

FIG. 17 is a timing chart showing timings, with which light beams having wavelengths falling within different wavelength regions are irradiated in the fluorescence endoscope system of FIG. 14,

FIG. 18 is an explanatory view showing an image of living body tissues detected by the fluorescence endoscope system of FIG. 14,

FIG. 19 is an explanatory view showing a moving average filter,

FIG. 20 is an explanatory view showing a differentiation filter,

FIG. 21 is an explanatory view showing a luminous point and a surrounding area,

FIG. 22A is an explanatory view showing an image of living body tissues detected from reflected light of light having been irradiated from a channel A,

FIG. 22B is an explanatory view showing an image of living body tissues detected from reflected light of light having been irradiated from a channel B,

FIG. 23 is a block diagram showing an operation processing unit employed in a fluorescence endoscope system, in which a second embodiment of the apparatus for acquiring an endoscope image in accordance with the present invention is employed,

FIG. 24A is an explanatory view showing values of pixels represented by a two-dimensional image signal, which has been detected from reflected light of light having been irradiated from the channel A,

FIG. 24B is an explanatory view showing values of pixels represented by a two-dimensional image signal, which has been detected from reflected light of light having been irradiated from the channel B,

FIG. 25 is an explanatory view showing values of pixels represented by a two-dimensional image signal, which has been obtained by subtracting the two-dimensional image signal of FIG. 24B from the two-dimensional image signal of FIG. 24A, and

FIG. 26 is an explanatory view showing a rotating filter provided with a filter for transmitting only near infrared light.

### DESCRIPTION OF THE PREFERRED EMBODIMENTS

The present invention will hereinbelow be described in further detail with reference to the accompanying drawings.

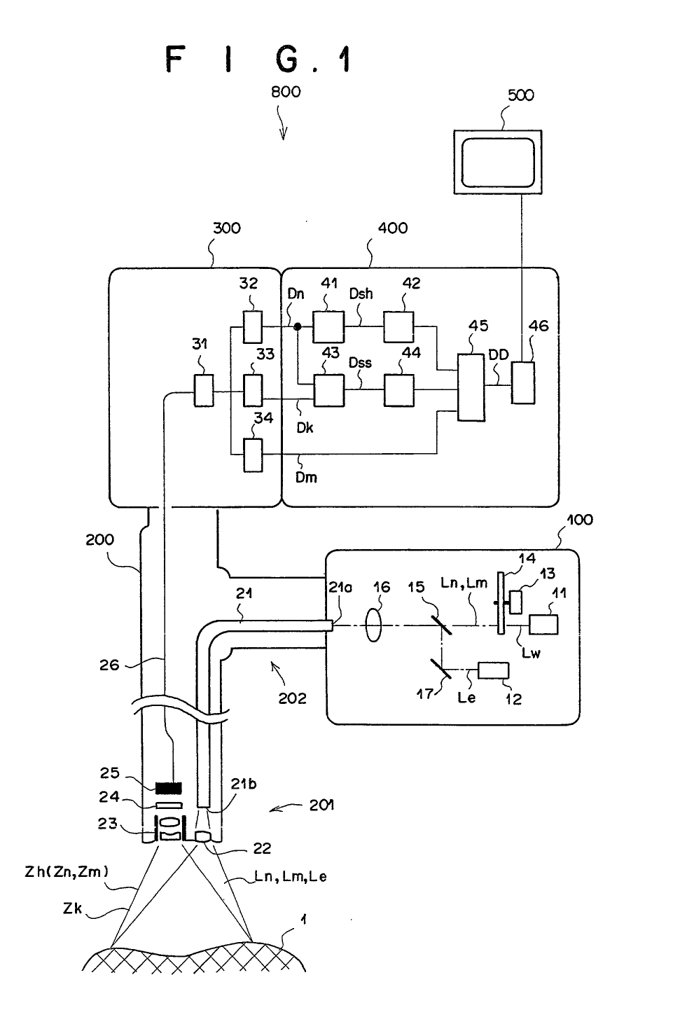

FIG. 1 is a schematic view showing a fluorescence endoscope system, in which a first embodiment of the apparatus for displaying a fluorescence image in accordance with the present invention is employed.

In a fluorescence endoscope system 800, in which the first embodiment of the apparatus for displaying a fluorescence image in accordance with the present invention is employed, operation processing is performed on a fluorescence image signal Dk and a reflected reference light image signal Dn. The fluorescence image signal Dk represents a first fluorescence image having been obtained by detecting fluorescence components of fluorescence having been produced from living body tissues 1 exposed to excitation light Le, which fluorescence components have wavelengths falling within a specific wavelength region. The reflected reference light image signal Dn represents a reflected reference light image having been obtained by detecting reflected reference light, which has been reflected from the living body tissues 1 when reference light Ln is irradiated to the living body tissues 1. With the operation processing, a tissue condition image signal DD representing a tissue condition image, which represents a tissue condition of the living body tissues 1 and which has been compensated for a distance to the living body tissues 1, is formed. In cases where the tissue condition image represented by the tissue condition image signal DD is to displayed, a judgment is made as to whether each of image areas embedded in the tissue condition image represented by the tissue condition image signal DD is an abnormal light affected area, which has been affected by light having an intensity equal to at least a specified value, or a normal light detection area, which has been formed with light having an intensity lower than the specified value. The judgment is made by a regularly reflected light area recognizer 41, which acts as the judgment means, and in accordance with either one of the first fluorescence image, which is represented by the fluorescence image signal Dk, and the reflected reference light image, which is represented by the reflected reference light image signal Dn. In accordance with an output of the regularly reflected light area recognizer 41 acting as the judgment means, a tissue condition image composer 45, which acts as the abnormal light affected area displaying means, displays the abnormal light affected area in a form different from the normal light detection area. The specified value is determined in accordance with an intensity of the reflected reference light, which intensity indicates the presence of regularly reflected light, in the reflected reference light image signal Dn. The abnormal light affected area, which has been affected by light having an intensity equal to at least the specified value, is judged as being a regularly reflected light area.

With reference to FIG. 1, the fluorescence endoscope system 800 comprises a light source unit 100 provided with two light sources for producing light having wavelengths falling with indifferent wavelength regions. The fluorescence endoscope system 800 also comprises an endoscope unit 200 for receiving the light from the light source unit 100, irradiating the light via an irradiating optical fiber 21, which will be described later, to living body tissues 1, and detecting an image, which is formed with reflected light having been reflected by the living body tissues 1 when the light is irradiated to the living body tissues 1, and an image, which is formed with fluorescence produced from the living body tissues 1. (The image formed with the reflected light having been reflected by the living body tissues 1 will hereinbelow be referred to as the reflected light image Zh. Also, the image formed with the fluorescence will hereinbelow be referred to as the fluorescence image Zk.) The fluorescence endoscope system 800 further comprises a relay unit 300 for converting the reflected light image Zh and the fluorescence image Zk, which have been detected by the endoscope unit 200, into two-dimensional image signals, which are constituted of digital values. The fluorescence endoscope system 800 still further comprises an operation processing unit 400, which is provided with the regularly reflected light area recognizer 41 and the tissue condition image composer 45. The operation processing unit 400 performs operation processing on the two-dimensional image signals, which have been received from the relay unit 300, and a judgment of the regularly reflected light area in order to obtain two-dimensional image signals representing the tissue condition of the living body tissues 1, and transforms the thus obtained two-dimensional image signals into video signals.

The light source unit 100 comprises a white light source 11 for producing the white light Lw, which has wavelengths falling within a near infrared wavelength region in the vicinity of 780 nm and a visible wavelength region. The light source unit 100 also comprises an excitation light source 12 for producing the excitation light Le, which has a wavelength of 410 nm. The white light Lw, which has been produced by the white light source 11, passes through a rotating filter 14, which comprises a combination of a plurality of filters having different wavelength transmission characteristics and is fitted to a main shaft of a motor 13. The light having passed through the rotating filter 14 passes through a dichroic mirror 15, which reflects light having wavelengths falling within a wavelength region of at most 410 nm and transmits only light having wavelengths falling within a wavelength region longer than 410 nm. The light having passed through the dichroic mirror 15 is converged by a converging lens 16 and impinges upon an end face 21a of the irradiating optical fiber 21. The excitation light Le, which has been produced by the excitation light source 12, is reflected by the dichroic mirror 15 and converged by the converging lens 16. The excitation light Le then impinges upon the end face 21a of the irradiating optical fiber 21.

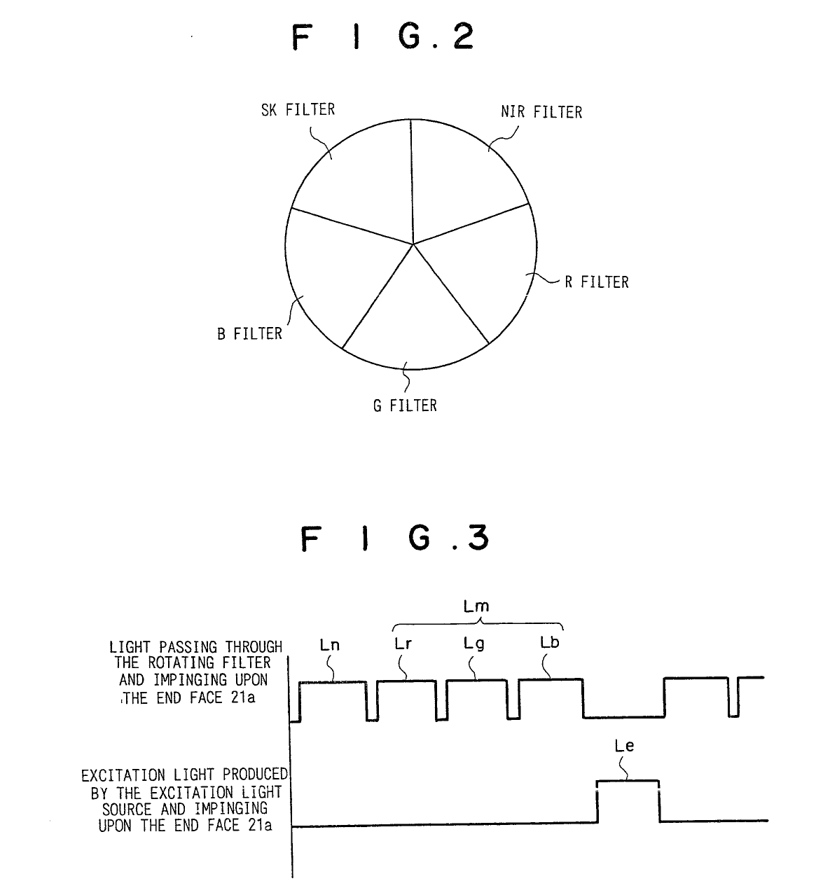

As illustrated in FIG. 2, the rotating filter 14 comprises an NIR filter, which transmits only light having wavelengths falling within the near infrared wavelength region, an R filter, which transmits only light having wavelengths falling within the red wavelength region, a G filter, which transmits only light having wavelengths falling within the green wavelength region, a B filter, which transmits only light having wavelengths falling within the blue wavelength region, and an SK filter (i.e., a light blocking filter), which blocks light. As illustrated in the timing chart of FIG. 3, when the rotating filter 14 rotates, the white light Lw having been produced by the white light source 11 is separated into near infrared light Ln, red light Lr, green light Lg, and blue light Lb. (The near infrared light Ln will hereinbelow be referred to as the reference light Ln. Also, the group of the red light Lr, the green light Lg, and the blue light Lb will hereinbelow be referred to as the surface sequential light Lm.) The near infrared light Ln, red light Lr, green light Lg, and blue light Lb, which have been separated from one another, successively impinge upon the end face 21a of the irradiating optical fiber 21. Also, when the white light Lw is being blocked by the SK filter, the excitation light Le, which has been produced by the excitation light source 12, is reflected by a mirror 17 and the dichroic mirror 15 and impinges upon the end face 21a of the irradiating optical fiber 21.

The endoscope unit 200 comprises a flexible leading end section 201 and a manipulating section 202, which is connected to the light source unit 100 and the relay unit 300. The irradiating optical fiber 21 extends from the leading end section 201 to the manipulating section 202 in the endoscope unit 200.

The reference light Ln, the surface sequential light Lm, and the excitation light Le, which have impinged upon the end face 21a of the irradiating optical fiber 21, are guided through the irradiating optical fiber 21, radiated out from an end face 21b of the irradiating optical fiber 21, and irradiated through an irradiating lens 22 to the living body tissues 1.

An image of the living body tissues 1, which is formed with reflected reference light having been reflected by the living body tissues 1 when the reference light Ln is irradiated to the living body tissues 1, and an image of the living body tissues 1, which is formed with reflected surface sequential light having been reflected by the living body tissues 1 when the surface sequential light Lm is irradiated to the living body tissues 1, are formed by an objective lens 23 and on a light receiving surface of an image sensor 25. (The image formed with the reflected reference light will hereinbelow be referred to as the reflected reference light image Zn. Also, the image formed with the reflected surface sequential light will hereinbelow be referred to as the surface sequential light image Zm.) The reflected reference light image Zn and the surface sequential light image Zm are detected and converted by the image sensor 25 into electric image signals. The electric image signals are transmitted through a cable 26 into the relay unit 300. Also, a fluorescence image Zk formed with the fluorescence, which has been produced from the living body tissues 1 when the excitation light Le is irradiated to the living body tissues 1 and which has wavelengths falling within a wavelength region of a value longer than 410 nm to a value in the vicinity of 700 nm, is formed by the objective lens 23 and on the light receiving surface of the image sensor 25. The fluorescence image Zk is detected and converted by the image sensor 25 into an electric image signal. The thus obtained electric image signal is transmitted through the cable 26 into the relay unit 300. An excitation light cut-off filter 24, which filters out light having a wavelength of 410 nm and transmits only light having wavelengths falling within the wavelength region longer than 410 nm, is located between the objective lens 23 and the image sensor 25. Reflected excitation light (i.e., reflected light of the excitation light), which is mixed in the fluorescence image Zk and has impinged upon the objective lens 23, is filtered out by the excitation light cut-off filter 24.

The relay unit 300 comprises an analog-to-digital converter 31 for converting each of the image signals, which have been transmitted through the cable 26, into a digital image signal. The relay unit 300 also comprises a reflected reference light image memory 32 for storing the two-dimensional image signal, which represents the reflected reference light image Zn and has been received from the analog-to-digital converter 31, as the reflected reference light image signal Dn. The relay unit 300 further comprises a fluorescence image memory 33 for storing the two-dimensional image signal, which represents the fluorescence image Zk and has been received from the analog-to-digital converter 31, as the fluorescence image signal Dk. The relay unit 300 still further comprises a surface sequential light image memory 34 for storing the two-dimensional image signal, which represents the surface sequential light image Zm and has been received from the analog-to-digital converter 31, as a surface sequential light image signal Dm.

The operation processing unit 400 comprises the regularly reflected light area recognizer 41 for receiving the reflected reference light image signal Dn and recognizing a regularly reflected light area having been affected by regularly reflected light, which area is embedded in the image represented by the reflected reference light image signal Dn. A regularly reflected light area signal Dsh, which represents the recognized regularly reflected light area, is obtained from the regularly reflected light area recognizer 41. The operation processing unit 400 also comprises a regularly reflected light area memory 42 for storing the regularly reflected light area signal Dsh having been received from the regularly reflected light area recognizer 41. The operation processing unit 400 further comprises a fluorescence yield calculator 43 for receiving the reflected reference light image signal Dn and the fluorescence image signal Dk and forming a fluorescence yield image signal Dss, which represents the tissue condition of the living body tissues 1, from the received signals. The operation processing unit 400 still further comprises a fluorescence yield image memory 44 for storing the fluorescence yield image signal Dss having been received from the fluorescence yield calculator 43. The regularly reflected light area signal Dsh having been stored in the regularly reflected light area memory 42, the fluorescence yield image signal Dss having been stored in the fluorescence yield image memory 44. and the surface sequential light image signal Dm having been stored in the surface sequential light image memory 34 are fed into the tissue condition image composer 45. In the tissue condition image composer 45, the regularly reflected light area signal Dsh, the fluorescence yield image signal Dss, and the surface sequential light image signal Dm are superimposed one upon another so as to form a composed image signal representing one image. The composed image signal is then transformed by a video signal processing circuit 46 into video signals.

The video signals are fed from the operation processing unit 400 into a display device 500 and utilized for displaying a visible image.

How the fluorescence endoscope system, in which the first embodiment of the apparatus for displaying a fluorescence image in accordance with the present invention is employed, operates will be described hereinbelow. In this embodiment, in order for a fluorescence image to be obtained, the excitation light Le having a wavelength of 410 nm is irradiated to the living body tissues 1. Also, in order for a reflected reference light image to be obtained, the near infrared light having a wavelength of 780 nm is irradiated as the reference light Ln to the living body tissues 1. Further, in order for the color and the shape of the living body tissues 1 to be seen, the surface sequential light Lm is irradiated to the living body tissues 1.

The excitation light Le, which causes the living body tissues 1 to produce the fluorescence, is radiated out from the light source unit 100 and irradiated via the endoscope unit 200 to the living body tissues 1. The fluorescence image Zk of the living body tissues 1, which is formed with the fluorescence having been produced from the living body tissues 1, is detected by the image sensor 25. Also, the reference light Ln and the surface sequential light Lm are radiated out from the light source unit 100 and irradiated via the endoscope unit 200 to the living body tissues 1. The reflected reference light image Zn of the living body tissues 1, which is formed with the reflected reference light having been reflected by the living body tissues 1 when the reference light Ln is irradiated to the living body tissues 1, and the surface sequential light image Zm of the living body tissues 1, which is formed with the reflected surface sequential light having been reflected by the living body tissues 1 when the surface sequential light Lm is irradiated to the living body tissues 1, are detected by the image sensor 25. The image signals representing the fluorescence image Zk, the reflected reference light image Zn, and the surface sequential light image Zm are transmitted into the relay unit 300 and converted into the two-dimensional image signals, which are constituted of digital values. The two-dimensional image signal representing the fluorescence image Zk is stored in the fluorescence image memory 33. The two-dimensional image signal representing the reflected reference light image Zn is stored in the reflected reference light image memory 32. Also, the two-dimensional image signal representing the surface sequential light image Zm is stored in the surface sequential light image memory 34.

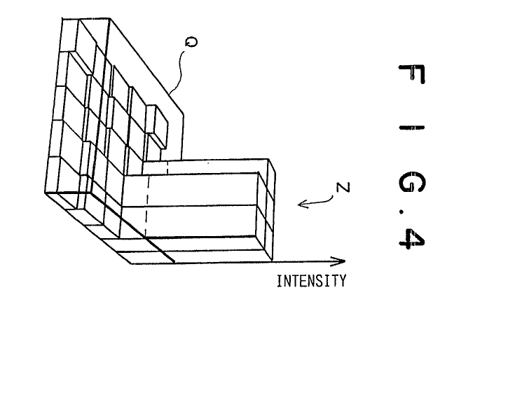

The reflected reference light image signal Dn, which represents the reflected reference light image Zn and has been stored in the reflected reference light image memory 32, is fed into the regularly reflected light area recognizer 41. In the regularly reflected light area recognizer 41, a pixel area represented by the reflected reference light image signal Dn, which area corresponds to an area having a markedly high intensity in the reflected reference light image Zn, i.e. a pixel area Z having an intensity higher than a predetermined threshold value Q among the intensities at respective pixel positions as illustrated in FIG. 4, is recognized as the regularly reflected light area. The regularly reflected light area signal Dsh representing the recognized regularly reflected light area is stored in the regularly reflected light area memory 42.

Also, the reflected reference light image signal Dn, which represents the reflected reference light image Zn and has been stored in the reflected reference light image memory 32, and the fluorescence image signal Dk, which represents the fluorescence image Zk and has been stored in the fluorescence image memory 33, are fed into the fluorescence yield calculator 43. In the fluorescence yield calculator 43, signal values of the fluorescence image signal Dk and the reflected reference light image signal Dn, which signal values represent corresponding pixels in the fluorescence image Zk and the reflected reference light image Zn, are divided by each other (i.e., the ratio of the signal value of the fluorescence image signal Dk to the signal value of the reflected reference light image signal Dn is calculated),and the fluorescence yield image signal Dss is thereby obtained. Specifically, the division represented by the formula shown below is performed with respect to each of the pixels, and the values of the fluorescence yield image signal Dss are calculated.

Dss=Dk/Dn

The fluorescence yield image signal Dss is equivalent to a two-dimensional image signal representing the fluorescence yield that is the ratio of the intensity of the fluorescence, which has been produced from the living body tissues 1 when the excitation light Le is irradiated to the living body tissues 1, to the intensity of the excitation light Le, which is received by the living body tissues 1. Specifically, since it is not easy to directly measure the distribution of the intensity of the excitation light Le, which is received by the living body tissues 1, the distribution of the intensity of the reflected reference light having been reflectedby the living body tissues 1 is utilized in lieu of the distribution of the intensity of the excitation light Le, which is received by the living body tissues 1, and the fluorescence yield is thereby calculated. The fluorescence yield image signal Dss is stored in the fluorescence yield image memory 44.

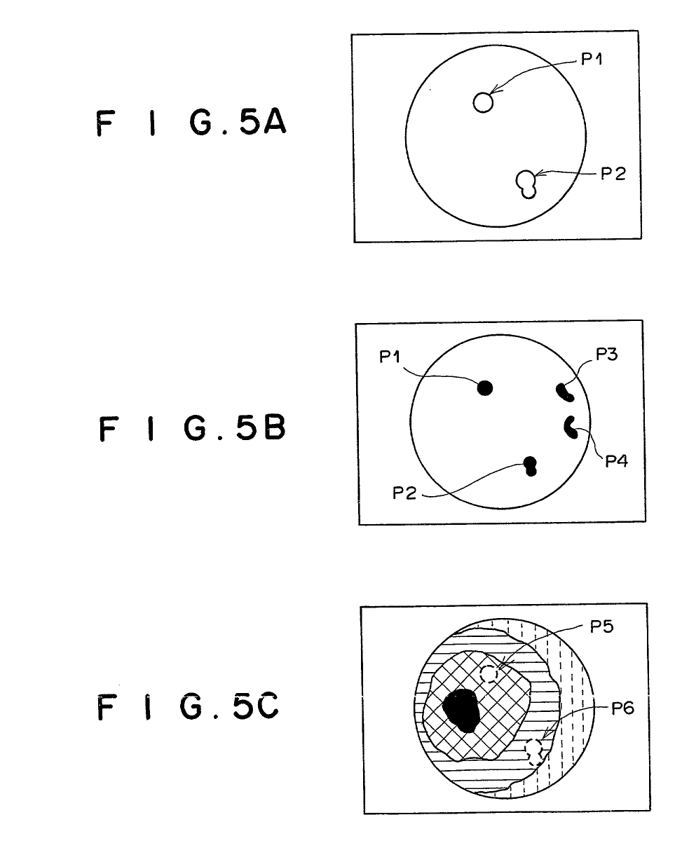

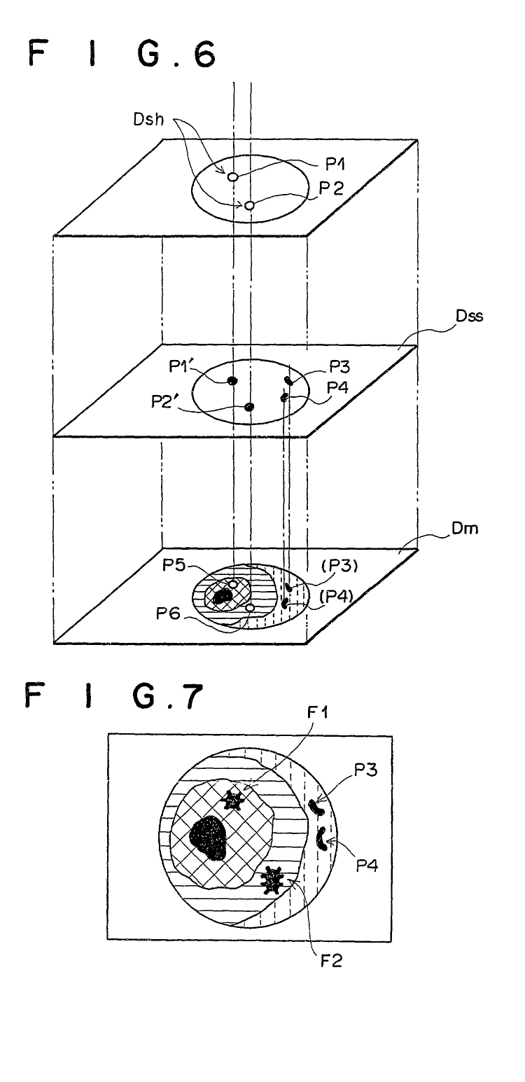

Thereafter, the regularly reflected light area signal Dsh, the fluorescence yield image signal Dss, and the surface sequential light image signal Dm, which have been obtained in the manner described above, are fed into the tissue condition image composer 45. As illustrated in FIG. 5A, the regularly reflected light area signal Dsh represents areas P1 and P2, at which the reference light Ln has been regularly reflected from the living body tissues 1. The fluorescence yield image signal Dss represents the tissue condition of the living body tissues 1. Specifically, as illustrated in FIG. 5B, the fluorescence yield image signal Dss represents diseased tissue areas P3 and P4. The fluorescence yield image signal Dss also contains signal components representing areas P1′ and P2′, which have been affected by the regularly reflected light and are displayed in a form approximately identical with the form of the diseased tissues due to the effects of the regularly reflected light. As illustrated in FIG. 5C, the surface sequential light image signal Dm represents the color and the shape of the living body tissues 1, which color and shape are seen ordinarily. In FIG. 5C, P5 and P6 are the areas, which appear as luminous points due to the regular reflection of the surface sequential light Lm from the living body tissues 1.

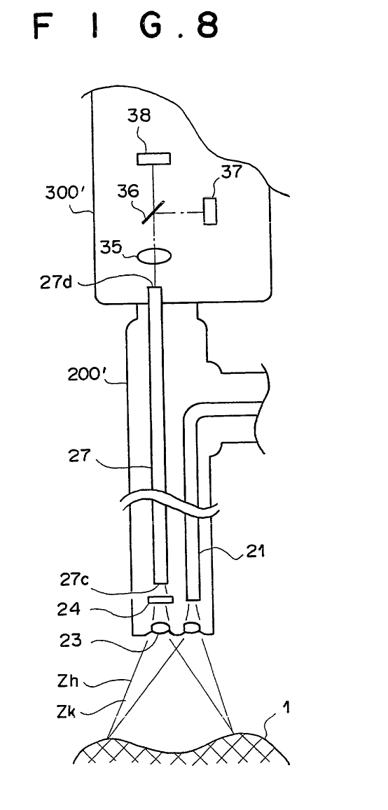

As illustrated in FIG. 6, when the three kinds of the signals described above are fed into the tissue condition image composer 45, the image, in which the areas P3 and P4 having been recognized as the diseased tissue areas in accordance with the fluorescence yield image signal Dss have been embedded, (i.e., the image in which the normal tissue areas have values close to 0 and the diseased tissue areas have large values) is added onto the living body tissue image, which is an ordinarily seen image and is represented by the surface sequential light image signal Dm, (i.e., the image in which the bright areas have values close to 0 and the dark areas have large values). Also, an image is composed as illustrated in FIG. 7. In the composed image illustrated in FIG. 7, the areas corresponding to the areas P1 and P2 represented by the regularly reflected light area signal Dsh, i.e. the areas overlapping upon the areas P5 and P6 represented by the surface sequential light image signal Dm and the areas P1′ and P2′ represented by the fluorescence yield image signal Dss, are displayed in specific regularly reflected light area displaying forms F1 and F2, which have been determined previously, (i.e., in the displaying forms in which the peripheries of the areas have protrusions and the insides of the areas are dark), such that the areas corresponding to the areas P1 and P2 are capable of being clearly discriminated from the regions P3 and P4, which have been recognized as being the diseased tissue areas. From the tissue condition image composer 45, the tissue condition image signal DD representing the composed image is obtained.

The tissue condition image signal DD is transformed by the video signal processing circuit 46 into video signals. The video signals are fed from the operation processing unit 400 into the display device 500 and utilized for displaying a visible image. The specific regularly reflected light area displaying form, which has been determined previously, for representing the regularly reflected light areas may be selected from various displaying forms such that the tissue condition of the living body tissues 1 is capable of being discriminated from the diseased tissues. For example, in lieu of the specific regularly reflected light area displaying forms F1 and F2 described above, displaying forms maybe employed, wherein the regularly reflected light areas are surrounded by frames and the luminous points due to the regularly reflected light, which luminous points are embedded in the image represented by the surface sequential light image signal Dm, are seen within the frames. In the displayed image, even if the leading end section 201 of the endoscope unit 200 is being moved, the regularly reflected light areas can be displayed in the specific regularly reflected light area displaying forms F1 and F2, which have been determined previously, together with the image representing the tissue condition of the living body tissues 1. Therefore, the tissue condition of the living body tissues 1 is capable of being seen with a high reliability.

When the tissue condition image is being seen as a dynamic image, the processing for displaying the regularly reflected light areas in the specific displaying forms may not be performed. only when the tissue condition image is to be seen as a still image, the processing for displaying the regularly reflected light areas in the specific displaying forms may be performed.

Also, the tissue condition image signal DD representing the tissue condition may be formed by utilizing the two kinds of the signals, i.e. the regularly reflected light area signal Dsh representing the regularly reflected light areas and the fluorescence yield image signal Dss representing the tissue condition of the living body tissues 1. In such cases, as illustrated in FIG. 8, the fluorescence image Zk and the reflected reference light image Zn may be passed through the objective lens 23 and the excitation light cut-off filter 24 and may then be formed on an end face 27c of an image fiber 27. The fluorescence image Zk and the reflected reference light image Zn may then be guided through the image fiber 27 to an end face 27d of the image fiber 27 and passed through an image forming lens 35 and a dichroic mirror 36 for separating light of a wavelength region of visible light and light of a near infrared region from each other. The fluorescence image Zk and the reflected reference light image may thus be separated from each other with respect to the wavelength regions and may be formed on an image sensor 37 and an image sensor 38. In this manner, the image signals may be obtained.

The tissue condition image may be one of various kinds of images, which are obtained in accordance with the fluorescence image representing the tissue condition of the living body tissues and the reflected reference light image representing the regularly reflected light area. For example, as the reflected reference light image representing the regularly reflected light area, a reflected reference light image representing the regularly reflected light area and formed by irradiating the excitation light, which has a wavelength of 410 nm, or reference light, which has wavelengths falling within the red wavelength region, to the living body tissues 1 may be employed. Also, as the fluorescence image representing the tissue condition of the living body tissues, for example, a fluorescence image representing the normalized fluorescence intensity having been calculated from division of the intensity of fluorescence components of the fluorescence having been produced from the living body tissues 1 exposed to the excitation light, which fluorescence components have wavelengths falling within a specific wavelength region, by the intensity of the fluorescence components, which have wavelengths falling within the entire wavelength region of the fluorescence, may be employed. In this manner, the tissue condition image may be obtained. However, in order for the normalized fluorescence intensity to be calculated, it is necessary to utilize an optical system for separating the fluorescence components, which have wavelengths falling within the specific wavelength region, from the fluorescence image and detecting the thus separated fluorescence components.

Further, in lieu of the technique for recognizing the regularly reflected light area in the manner described above, the regularly reflected light area may be recognized by employing image processing with an differentiation operator, or the like.

Besides the fluorescence endoscope system described above, the method and apparatus for displaying a fluorescence image in accordance with the present invention are also applicable to colposcopes, operating microscopes, and the like.

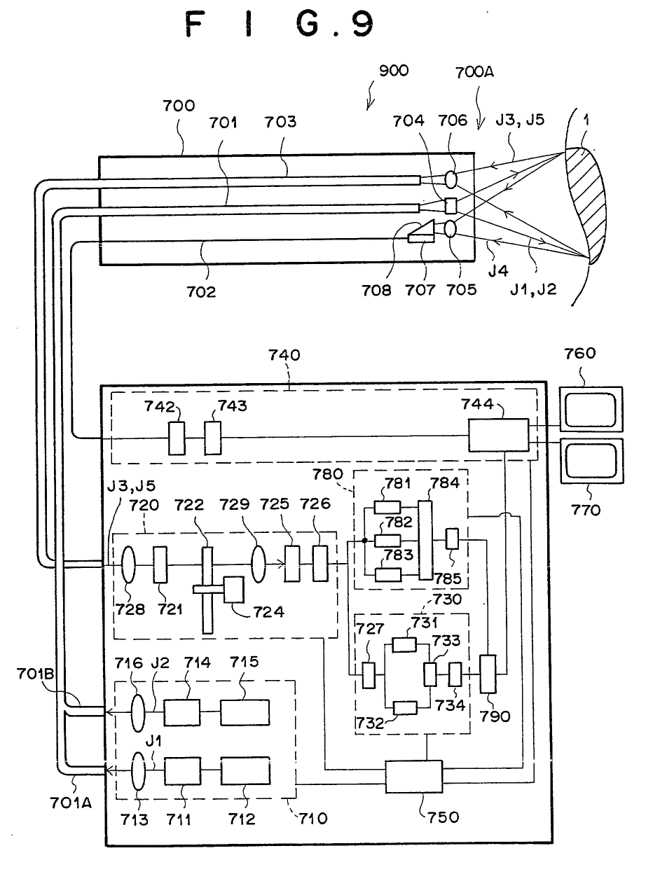

A fluorescence endoscope system, in which a second embodiment of the apparatus for displaying a fluorescence image in accordance with the present invention is employed, will be described hereinbelow with reference to FIG. 9.

In a fluorescence endoscope system 900, in which the second embodiment of the apparatus for displaying a fluorescence image in accordance with the present invention is employed, operation processing is performed on a narrow-band fluorescence image, a broad-band fluorescence image, and an IR reflected reference light image. The narrow-band fluorescence image is a second fluorescence image having been obtained by detecting fluorescence components of fluorescence having been produced from living body tissues exposed to excitation light, which fluorescence components have wavelengths falling within a specific wavelength region of 430 nm to 530 nm. The broad-band fluorescence image is a first fluorescence image having been obtained by detecting fluorescence components of the fluorescence, which fluorescence components have wavelengths falling within a wavelength region of 430 nm to 730 nm different from the specific wavelength region described above. The IR reflected reference light image is a reflected reference light image having been obtained by detecting light components of light having been reflected from the living body tissues when white light containing near infrared light acting as reference light is irradiated to the living body tissues, which light components have wavelengths falling within a near infrared wavelength region of 750 nm to 900 nm. With the operation processing, a tissue condition image, which represents a tissue condition of the living body tissues and which has been compensated for a distance to the living body tissues, is formed. In cases where the tissue condition image is to displayed, a judgment is made as to whether each of image areas embedded in the tissue condition image is an abnormal light affected area, which has been affected by light having an intensity equal to at least a specified value, or a normal light detection area, which has been formed with light having an intensity lower than the specified value. The judgment is made by an image judgment unit 780, which acts as the judgment means, and in accordance with the narrow-band fluorescence image acting as the second fluorescence image, the broad-band fluorescence image acting as the first fluorescence image, and the IR reflected reference light image acting as the reflected reference light image. In accordance with an output of the image judgment unit 780, an image composer 790, which acts as the abnormal light affected area displaying means, displays the abnormal light affected area in a form different from the normal light detection area. The specified value is determined in accordance with a limit of detection in the IR reflected reference light image and limits of effective measurement ranges in the narrow-band fluorescence image and the broad-band fluorescence image.

With reference to FIG. 9, the fluorescence endoscope system 900 comprises an endoscope tube 700 to be inserted into the living body. The fluorescence endoscope system 900 also comprises an illuminating unit 710 provided with a white light source for producing light, which has wavelengths falling within a visible wavelength region and the near infrared wavelength region, and an excitation light source for producing the excitation light, which has a wavelength in the vicinity of 410 nm and which excites the living body tissues to produce the fluorescence. The fluorescence endoscope system 900 further comprises an imaging unit 720 for detecting an image, which is formed with the fluorescence having been produced from the living body tissues, and an image, which is formed with the near infrared light having been reflected from the living body tissues. The fluorescence endoscope system 900 still further comprises a tissue condition image forming unit 730 for forming the tissue condition image, which represents the tissue condition of the living body tissues, in accordance with the image having been detected by the imaging unit 720. The fluorescence endoscope system 900 also comprises an ordinary image processing unit 740 for performing signal processing for displaying an ordinary image, which has been detected by an image sensor located within the endoscope tube 700 and which is equivalent to a visually obtained image. The fluorescence endoscope system 900 further comprises a controller 750, which are connected to the respective units described above and which controls operation timings, and a video monitor 760 for displaying the ordinary image, which has been obtained from the image processing performed by the ordinary image processing unit 740, as a visible image. The fluorescence endoscope system 900 still further comprises the image judgment unit 780 acting as the judgment means, which receives the image having been detected by the imaging unit 720 and makes a judgment as to whether each of areas in the image is the abnormal light affected area or the normal light detection area. The fluorescence endoscope system 900 also comprises the image composer 790 acting as the abnormal light affected area displaying means, which receives the tissue condition image from the tissue condition image forming unit 730 and receives the results of the judgment from the image judgment unit 780. The image composer 790 displays the abnormal light affected area, which is embedded in the tissue condition image, in a form different from the normal light detection area. The fluorescence endoscope system 900 further comprises a video monitor 770 for receiving a composed image from the image composer 790 via a video signal forming circuit 744 of the ordinary image processing unit 740 and displaying the composed image as a visible image.

A light guide 701, a CCD (charge coupled device) cable 702, and an image fiber 703 extend in the endoscope tube 700. An illuminating lens 704 is located in front of an end face of the light guide 701. A converging lens 706 is located in front of an end face of the image fiber 703, which is constituted of quartz glass fibers. A CCD image sensor 707, which is combined with a color mosaic filter, is connected to one end of the CCD cable 702. A prism 708 is located such that it is in close contact with the CCD image sensor 707. The light guide 701 comprises a white light guide 701A, which is constituted of a compound glass fiber, and an excitation light guide 701B, which is constituted of a quartz glass fiber. The white light guide 701B and the excitation light guide 701B are bundled together in a cable-like form to constitute the light guide 701. A tail end of the light guide 701, which tail end is located on the side outward from the endoscope tube 700, is connected to the illuminating unit 710. Also, a tail end of the CCD cable 702, which tail end is located on the side outward from the endoscope tube 700, is connected to the ordinary image processing unit 740. A tail end of the image fiber 703 is connected to the imaging unit 720.

The illuminating unit 710 comprises a white light source 711 for producing white light J1, and an electric power source 712 for feeding electric power to the white light source 711. The illuminating unit 710 also comprises a GaN type of semiconductor laser 714 for producing excitation light J2, which is used when the fluorescence image is to be displayed, and an electric power source 715 for feeding electric power to the GaN type of semiconductor laser 714.

The imaging unit 720 comprises an excitation light cut-off filter 721 for filtering out light, which has wavelengths falling within a wavelength region of at most 420 nm containing the wavelength region of the excitation light J2, from fluorescence J3 having passed through the image fiber 703. The imaging unit 720 also comprises a rotating filter 722 constituted of three kinds of optical filters, which have different wavelength characteristics and which have been combined into an integral body. The imaging unit 720 further comprises a filter rotating device 724 for rotating the rotating filter 722. The imaging unit 720 still further comprises a CCD image sensor 725 for detecting the fluorescence image or the IR reflected reference light image having passed through the rotating filter 722. The imaging unit 720 also comprises an analog-to-digital converting circuit 726 for digitizing a signal, which has been obtained from the CCD image sensor 725.

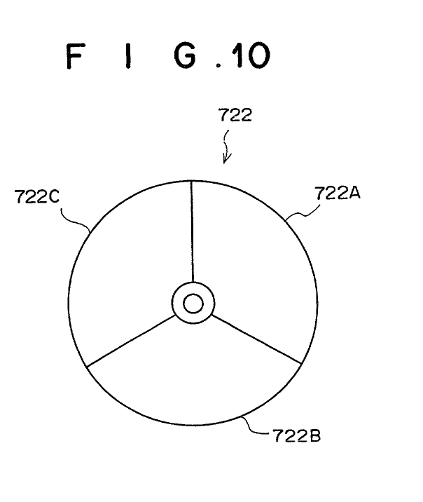

As illustrated in FIG. 10, the rotating filter 722 comprises a broad band-pass filter 722A for transmitting light having wavelengths falling within the wavelength region of 430 nm to 730 nm, a narrow band-pass filter 722B for transmitting light having wavelengths falling within the wavelength region of 430 nm to 530 nm, and an IR band-pass filter 722C for transmitting light having wavelengths falling within the wavelength region of 750 nm to 900 nm. The broad band-pass filter 722A is the filter for the detection of the broad-band fluorescence image. The narrow band-pass filter 722B is the filter for the detection of the narrow-band fluorescence image. The IR band-pass filter 722C is the filter for the detection of the IR reflected reference light image. The filter rotating device 724 is controlled by the controller 750 such that, when the white light is being irradiated to the living body tissues 1, the IR band-pass filter 722C of the rotating filter 722 is located in the optical path of the white light J1. The filter rotating device 724 is also controlled such that, when the excitation light J2 is being irradiated to the living body tissues 1, the broad band-pass filter 722A and the narrow band-pass filter 722B of the rotating filter 722 are successively located in the optical path of the excitation light J2.

The CCD image sensor 725 is constituted of 500×500 pixels. Under the control of the controller 750, when the IR reflected reference light image is to be detected, the CCD image sensor 725 performs an ordinary reading operation. Also, when the fluorescence image is to be detected, the CCD image sensor 725 performs a binning reading operation, in which outputs of 5×5 pixels are added together, and the thus obtained sum is read such that the amount of light received per pixel of the fluorescence image may be enhanced. Therefore, when the fluorescence image is to be detected, the CCD image sensor 725 apparently operates as an image sensor having 100×100 pixels.

As described above, the CCD image sensor 725 employs different reading techniques between when the IR reflected reference light image is to be detected and when the fluorescence image is to be detected. Therefore, the number of pixels constituting the IR reflected reference light image is 500×500, and the number of pixels constituting each of the narrow-band fluorescence image and the broad-band fluorescence image is 100×100.

The tissue condition image forming unit 730 comprises an image memory 727 for storing three kinds of image signals (representing the narrow-band fluorescence image, the broad-band fluorescence image, and the IR reflected reference light image), which have been detected through the rotating filter 722 and have been digitized by the analog-to-digital converting circuit 726. The tissue condition image forming unit 730 also comprises a color operation processing section 731 for performing a division between the two kinds of the fluorescence images (i.e., calculating the ratio between the two kinds of the fluorescence images) to find the normalized fluorescence intensity, finding correspondence relationship between the value of the normalized fluorescence intensity and a color by utilization of a look-up table having been stored previously, and transforming the value of the normalized fluorescence intensity into chrominance signals for the displaying of the visible image. The tissue condition image forming unit 730 further comprises a luminance operation processing section 732 for finding correspondence relationship between the value of the IR reflected reference light image and a luminance by utilization of a look-up table having been stored previously, and transforming the value of the IR reflected reference light image into a luminance signal for the displaying of the visible image. The tissue condition image forming unit 730 still further comprises a tissue condition image forming section 733 for forming the tissue condition image from the chrominance signals and the luminance signal, and a tissue condition image memory 734 for storing the image signal representing the tissue condition image.

Though not shown, the image memory 727 is constituted of a narrow-band fluorescence image storing region, a broad-band fluorescence image storing region, and an IR reflected reference light image storing region. The fluorescence image, which has been detected with the broad band-pass filter 722A being located in the optical path when the excitation light J2 is irradiated to the living body tissues 1, is converted by the analog-to-digital converting circuit 726 into the digital value, and the thus obtained image signal representing the broad-band fluorescence image is stored in the broad-band fluorescence image storing region. Also, the fluorescence image, which has been detected with the narrow band-pass filter 722B being located in the optical path when the excitation light J2 is irradiated to the living body tissues 1, is converted by the analog-to-digital converting circuit 726 into the digital value, and the thus obtained image signal representing the narrow-band fluorescence image is stored in the narrow-band fluorescence image storing region. Further, the IR reflected reference light image, which has been detected with the IR band-pass filter 722C being located in the optical path when the white light is irradiated to the living body tissues 1, is converted by the analog-to-digital converting circuit 726 into the digital value, and the thus obtained image signal representing the IR reflected reference light image is stored in the IR reflected reference light image storing region.

The image judgment unit 780 comprises an effective measurement range judging device 781 for making a judgment as to the area in the narrow-band fluorescence image, which area has been affected by light having an intensity equal to at least the specified value. The image judgment unit 780 also comprises an effective measurement range judging device 782 for making a judgment as to the area in the broad-band fluorescence image, which area has been affected by light having an intensity equal to at least the specified value. The image judgment unit 780 further comprises an overflow judging device 783 for making a judgment as to the area in the IR reflected reference light image, which area has been affected by light having an intensity equal to at least the specified value. The image judgment unit 780 still further comprises an abnormal light affected area judging device 784 for making a judgment as to the abnormal light affected area in accordance with the results of the judgments having been made by the three judging devices. The image judgment unit 780 also comprises an abnormal light affected area memory 785 for storing information representing the position of the abnormal light affected area, which information is obtained from the results of the judgment made as to the abnormal light affected area.

The image composer 790 receives the image signal representing the tissue condition image, which image signal has been stored in the tissue condition image memory 734, and the information representing the position of the abnormal light affected area, which information has been stored in the abnormal light affected area memory 785. The image composer 790 forms the composed image, in which the abnormal light affected area is illustrated in the tissue condition image.

The ordinary image processing unit 740 comprises an analog-to-digital converting circuit 742 for digitizing the image signal, which has been detected by the CCD image sensor 707, and an ordinary image memory 743 for storing the digital image signal representing the ordinary image. The ordinary image processing unit 740 also comprises the video signal forming circuit 744 for transforming the image signal representing the ordinary image, which image signal has been received from the ordinary image memory 743, and the image signal representing the composed image, which image signal has been received from the image composer 790, into video signals.

How the fluorescence endoscope system 900 operates will be described hereinbelow. Firstly, how the fluorescence endoscope system 900 operates when the ordinary image is to be detected and displayed will be described hereinbelow. Thereafter, how the fluorescence endoscope system 900 operates when the reflected reference light image and the fluorescence image are to be detected will be described. How the fluorescence endoscope system 900 operates when the composed image is to be formed and displayed will then be described.

In the fluorescence endoscope system 900, the detection of the ordinary image and the IR reflected reference light image and the detection of the fluorescence image are performed successively in the time division mode. When the ordinary image and the IR reflected reference light image are to be detected, the electric power source 712 is driven in accordance with a control signal fed from the controller 750, and the white light J1 containing the near infrared light, which acts as the reference light, is produced by the white light source 711. The white light J1 passes through a lens 713 and impinges upon the white light guide 701A. The white light J1 is then guided through the white light guide 701A to a leading end 700A of the endoscope tube 700 and is irradiated through the illuminating lens 704 to the living body tissues 1. The white light J1 impinging upon the living body tissues 1 is reflected as reflected light J4 by the living body tissues 1. The reflected light J4 of the white light J1 is converged by an objective lens 705 and reflected from the oblique surface of the prism 708. The reflected light J4 then passes through the color mosaic filter, and an image of the reflected light J4 is formed on the CCD image sensor 707. In this manner, the image of the reflected light J4 is detected as the ordinary image by the CCD image sensor 707. The ordinary image having been detected by the CCD image sensor 707 is converted by the analog-to-digital converting circuit 742 into the digital value, and the thus obtained digital image signal representing the ordinary image is stored in the ordinary image memory 743. The image signal having been stored in the ordinary image memory 743 is transformed by the video signal forming circuit 744 into the video signals, and the video signals are utilized for displaying the visible image on the video monitor 760. The series of the operations described above are controlled by the controller 750.

Also, reflected light J5 of the white light j1 containing the near infrared light is reflected from the living body tissues 1 and converged by the converging lens 706. The reflected light J5 impinges upon the end face of the image fiber 703, is guided through the image fiber 703, and is converged by a lens 728. The reflected light j5 then passes through the excitation light cut-off filter 721 and the IR band-pass filter 722C of the rotating filter 722, and an image of the reflected light J5 is formed as the IR reflected reference light image on the CCD image sensor 725.

The IR band-pass filter 722C is the band-pass filter for transmitting only the light having wavelengths falling within the wavelength region of 750 nm to 900 nm. Therefore, when the reflected light J5 passes through the IR band-pass filter 722C, only the reflected reference light is extracted, and only the IR reflected reference light image is formed on the CCD image sensor 725.

The IR reflected reference light image, which has been formed on the CCD image sensor 725 and detected, is photoelectrically converted into an image signal. The image signal is converted by the analog-to-digital converting circuit 726 into the digital signal, and the thus obtained digital signal is stored in the IR reflected reference light image storing region of the image memory 727.

How the fluorescence endoscope system 900 operates when the fluorescence image is to be detected will be described hereinbelow.

When the fluorescence image is to be detected, the electric power source 715 is driven in accordance with a control signal fed from the controller 750, and the excitation light J2 having a wavelength of 410 nm is produced by the GaN type of semiconductor laser 714. The excitation light J2 passes through a lens 716 and impinges upon the excitation light guide 701B. The white light J1 is then guided through the excitation light guide 701B to the leading end 700A of the endoscope tube 700 and is irradiated through the illuminating lens 704 to the living body tissues 1.

When the excitation light J2 is irradiated to the living body tissues 1, the fluorescence J3 is produced from the living body tissues 1. The fluorescence J3 is converged by the converging lens 706 and impinges upon the leading end of the image fiber 703. The fluorescence J3 is guided through the image fiber 703, is converged by the lens 728, then passes through the excitation light cut-off filter 721. Thereafter, the fluorescence J3 is transmitted successively through the broad band-pass filter 722A and the narrow band-pass filter 722B in the time division mode.

The fluorescence J3 having passed through the broad band-pass filter 722A and the fluorescence J3 having passed through the narrow band-pass filter 722B are successively received by the CCD image sensor 725 in the time division mode and subjected to photoelectric conversion and binning reading operation. With the binning reading operation, signal values of 5×5 pixels are added together, and the thus obtained sum is read. The thus obtained image signals are converted by the analog-to-digital converting circuit 726 into digital signals. The digital signal representing the broad-band fluorescence image is stored in the broad-band fluorescence image storing region of the image memory 727. Also, the digital signal representing the narrow-band fluorescence image is stored in the narrow-band fluorescence image storing region of the image memory 727. In cases where the binning reading operation is performed, the fluorescence image of a weak light intensity is capable of being detected accurately. However, with the binning reading operation, the number of the pixels constituting the image having been detected becomes equal to 100×100 pixels, i.e. 1/25 as large as the number of the pixels obtained in cases where the ordinary reading operation is performed.

How the composed image is formed will be described hereinbelow.

Firstly, the color operation processing section 731 of the tissue condition image forming unit 730 receives the image signals representing the narrow-band fluorescence image and the broad-band fluorescence image from the image memory 727. In the color operation processing section 731, the value of the narrow-band fluorescence image, which value represents a pixel in the narrow-band fluorescence image, is divided by the value of the broad-band fluorescence image, which value represents the corresponding pixel in the broad-band fluorescence image. In this manner, the normalized fluorescence intensity is calculated. Also, reference is made to a color look-up table having been stored previously in the color operation processing section 731, and the value of the normalized fluorescence intensity is transformed into chrominance signal components. Thereafter, the chrominance signal components corresponding to one pixel are transformed into chrominance signal components corresponding to 5×5 pixels. In this manner, the number of the pixels is restored from 100×100 pixels to 500×500 pixels, and the chrominance signals representing 500×500 pixels are obtained.

The luminance operation processing section 732 receives the image signal representing the IR reflected reference light image, which image signal has been stored in the IR reflected reference light image storing region of the image memory 727. In the luminance operation processing section 732, reference is made to a luminance look-up table having been stored previously in the image memory 727, and the value of the IR reflected reference light image, which value represents each pixel in the IR reflected reference light image, is transformed into a luminance signal component. The luminance signal made up of the thus obtained luminance signal components is obtained.

The tissue condition image forming section 733 receives the chrominance signals and the luminance signal described above and forms the image signal, which represents the tissue condition image, from the received signals. The image signal representing the tissue condition image is stored in the tissue condition image memory 734.

How the image judgment unit 780 and the image composer 790 operate will be described hereinbelow.

As described above, the image signals, which represent the narrow-band fluorescence image, the broad-band fluorescence image, and the IR reflected reference light image and which have been obtained from the analog-to-digital conversion performed by the analog-to-digital converting circuit 726, are fed into the image memory 727. Also, the image signals, which represent the narrow-band fluorescence image, the broad-band fluorescence image, and the IR reflected reference light image, are respectively fed into the effective measurement range judging device 781, the effective measurement range judging device 782, and the overflow judging device 783.

The image signal representing the narrow-band fluorescence image, which image signal has been fed into the effective measurement range judging device 781, and the image signal representing the broad-band fluorescence image, which image signal has been fed into the effective measurement range judging device 782, are compared with the specified values, which have been determined in accordance with the limits of the effective measurement ranges. In this manner, transfinite areas are determined. The specified values are determined previously with the techniques described below and stored in the effective measurement range judging device 781 and the effective measurement range judging device 782.

Specifically, the maximum light intensity, which is received when the fluorescence produced from the living body tissues 1 is detected with the fluorescence endoscope system 900, is the light intensity occurring when the fluorescence produced from the normal tissues of the living body is received in cases where the leading end 700A of the endoscope tube 700 of the fluorescence endoscope system 900 is set at the position closest to the living body tissues 1 in accordance with specifications of the fluorescence endoscope system 900. In cases where the tissue condition of the living body tissues 1 is seen with the fluorescence endoscope system 900, the limit of the distance between the leading end 700A and the living body tissues 1 has been determined to be 3 mm in accordance with the specifications of the fluorescence endoscope system 900. In cases where the distance between the leading end 700A and the living body tissues 1 is shorter than 3 mm, the tissue condition of the living body tissues 1 cannot be seen accurately.

Therefore, in cases where the leading end 700A of the endoscope tube 700 is set at the position close to the living body tissues 1, and the light intensity of the fluorescence received from the normal tissues is higher than the maximum light intensity, which is assumed to be received within the effective measurement range in accordance with the specifications of the fluorescence endoscope system 900, it is regarded that the distance between the leading end 700A and the living body tissues 1 has become shorter than 3 mm. The area associated with the light intensity higher than the maximum light intensity is regarded as the transfinite area, in which the tissue condition of the living body tissues 1 cannot be seen accurately.

The specified value for the determination of the transfinite area is determined by irradiating the excitation light to the living body tissues, which has been judged previously with a predetermined technique as being the normal tissues and which is located at a position spaced by a predetermined distance from the leading end 700A, detecting the intensity of the fluorescence having been produced from the living body tissues when the excitation light is irradiated to the living body tissues, and adding a value, which represents a variation of the detected values, to a mean value of the thus detected intensity values. Specifically, the specified value is determined by locating the leading end 700A of the endoscope tube 700 at the position spaced by 3 mm, which is the limit of approach in accordance with the specifications, from the normal tissues of the living body, iterating the irradiation of the excitation light to the normal tissues a plurality of times, measuring the intensities of the fluorescence produced from the normal tissues exposed to the excitation light, and calculating a mean value M and a standard deviation a of the measured intensities. The specified value is capable of being calculated with the formula E=M+2σ.

The specified value, which is stored in the effective measurement range judging device 781, has been determined by applying the technique described above to the detection of the fluorescence components of the fluorescence having been produced from the normal tissues, which fluorescence components have wavelengths falling within the wavelength region of 430 nm to 530 nm. The limit of the effective measurement range in the narrow-band fluorescence image is determined by the specified value having thus been determined. The specified value, which is stored in the effective measurement range judging device 782, has been determined by applying the technique described above to the detection of the fluorescence components of the fluorescence having been produced from the normal tissues, which fluorescence components have wavelengths falling within the wavelength region of 430 nm to 730 nm. The limit of the effective measurement range in the broad-band fluorescence image is determined by the specified value having thus been determined.

The image signal representing the IR reflected reference light image, which image signal has been fed into the overflow judging device 783, is compared with the specified value, which has been determined in accordance with the limit of the detection of the IR reflected reference light image, and the transfinite area is thereby determined. The limit of the detection of the IR reflected reference light image is determined as the one corresponding to the saturated value of the output of the image sensor for the detection of the IR reflected reference light image. The specified value in accordance with the limit of the detection has been determined previously with the technique described below and stored in the overflow judging device 783.

Specifically, the signal representing the IR reflected reference light image, which signal is obtained from the imaging unit 720, is the one obtained by converting the analog signal representing the IR reflected reference light image, which analog signal is obtained from the CCD image sensor 725, into the digital value with the analog-to-digital converting circuit 726. In cases where the value of the analog signal, which is received by the analog-to-digital converting circuit 726, (i.e., the detected intensity of the reflected reference light) is larger than the analog signal value, which the analog-to-digital converting circuit 726 is capable of converting, and saturation is reached in the digital output, the corresponding image area is regarded as an area, in which the tissue condition of the living body tissues cannot be seen accurately. Therefore, the saturated value of the digital output is determined as the specified value in accordance with the limit of the detection. For example, in cases where a 10-bit analog-to-digital converting circuit is utilized, the saturated value is equal to 1,024. The saturated value is determined as the specified value in the overflow judging device 783.

As illustrated in FIG. 11, in images H1, H2, and H3, transfinite areas U1, U1, . . . , transfinite areas U2, U2, . . . , and transfinite areas U3, U3, . . . are respectively embedded. The transfinite areas U1, U1, . . . , the transfinite areas U2, U2, . . . , and the transfinite areas U3, U3, . . . have been acquired respectively from the effective measurement range judging device 781, the effective measurement range judging device 782, and the overflow judging device 783 by making reference to the corresponding specified values. When the images H1, H2, and H3 are fed into the abnormal light affected area judging device 784, a logical product of the transfinite areas U1, U1, . . . , the transfinite areas U2, U2, . . . , and the transfinite areas U3, U3, . . . embedded in the images H1, H2, and H3 is calculated, and abnormal light affected areas U4, U4 are thereby determined. The information representing the positions of the abnormal light affected areas U4, U4, which have been determined by the abnormal light affected area judging device 784, is stored in the abnormal light affected area memory 785.

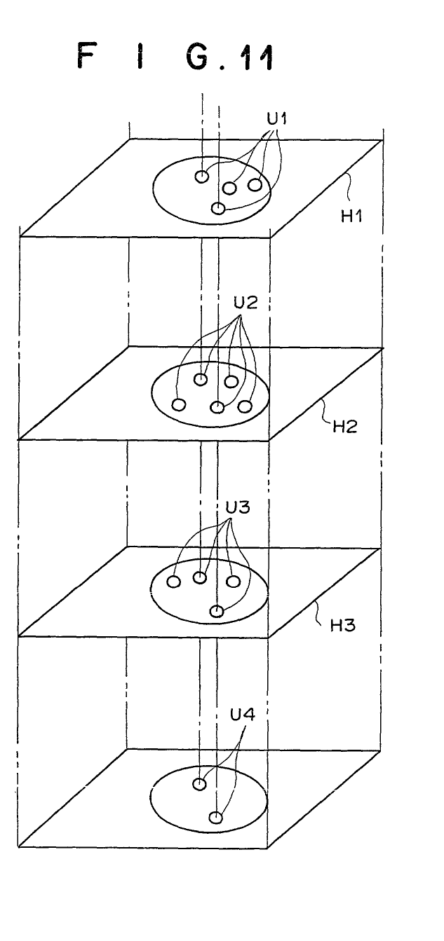

The image composer 790 receives the information representing the positions of the abnormal light affected areas U4, U4, which information has been stored in the abnormal light affected area memory 785, and the image signal representing the tissue condition image, which image signal has been stored in the tissue condition image memory 734. As illustrated in FIG. 12, the image composer 790 forms the composed image, such that abnormal light affected areas U4′, U4′ maybe illustrated as white areas in a tissue condition image S, which is displayed as a color image.

An image signal representing the thus composed image is fed from the image composer 790 into the video signal forming circuit 744. The image signal representing the composed image is transformed by the video signal forming circuit 744 into the video signals, and the video signals are utilized for displaying a visible composed image on the video monitor 770. The series of the operations described above are controlled by the controller 750.

The video signal forming circuit 744 performs both the signal processing on the composed image and the signal processing on the ordinary image, which is fed from the ordinary image memory 743.

In the visible composed image, which is displayed in the manner described above, the color represents the normalized fluorescence intensity, i.e. the diseased state of the living body tissues 1. Also, the luminance represents the intensity of the light having been reflected from the living body tissues 1, i.e. the shape of the living body tissues 1. Therefore, the information concerning the diseased state of the living body tissues 1 and the information concerning the shape of the living body tissues 1 are capable of being illustrated together on a single image.

Also, the abnormal light affected area, at which the tissue condition of the living body tissues 1 is not illustrated accurately, is illustrated as the white area in the image, which is displayed as the color image on the video monitor 770 and which represents the tissue condition of the living body tissues 1. Therefore, the problems are capable of being prevented from occurring in that the person, who sees the displayed image, makes an incorrect diagnosis by mistake. Accordingly, the tissue condition of the living body tissues 1 is capable of being seen with a high reliability.

Further, since the GaN type of semiconductor laser 714 is employed as the light source for producing the excitation light J2, the irradiation of the excitation light J2 is capable of being performed with the cheap, small-sized light source. Furthermore, since the wavelength of the excitation light J2 is 410 nm, the fluorescence is capable of being produced efficiently from the living body tissues 1.

In lieu of the normalized fluorescence intensity being utilized, the value of the fluorescence yield may be calculated by dividing the value of the pixel in the broad-band fluorescence image by the value of the corresponding pixel in the IR reflected reference light image. The value of the fluorescence yield may be allocated to the chrominance signal components. Also, the value of the pixel in the IR reflected reference light image may be allocated to the luminance signal component. In this manner, the tissue condition image may be formed.

Also, in the tissue condition image forming unit 730, the tissue condition image represented by the chrominance signals and the luminance signal need not necessarily be formed by utilizing both the color operation processing section 731 and the luminance operation processing section 732. For example, instead of the color operation processing section 731 being utilized, the value of the normalized fluorescence intensity, which has been calculated by dividing the value of the pixel in the narrow-band fluorescence image by the value of the corresponding pixel in the broad-band fluorescence image, or the value of the fluorescence yield, which has been calculated by dividing the value of the pixel in the broad-band fluorescence image by the value of the corresponding pixel in the IR reflected reference light image, maybe allocated to the luminance signal component, and the tissue condition image may thereby be formed. In this manner, the composed image may be formedby the image composer 790, such that the tissue condition image may be displayed as an achromatic, monochromatic image, and the abnormal light affected area may illustrated as a color area.

Further, the allocation of the value of the pixel in each of the images described above to the chrominance signal components or the luminance signal component may be performed such that a threshold value is set and the value of each pixel is binarized by the utilization of the threshold value so as to display a binary image. Alternatively, as in the embodiment described above, the values of the respective pixels may be allocated as continuous values, and the image may be displayed as a continuous change in color or luminance.

Furthermore, in the image judgment unit 780, the transfinite area may be determined by combining each of the images (i.e., the narrow-band fluorescence image acting as the second fluorescence image, the broad-band fluorescence image acting as the first fluorescence image, and the IR reflected reference light image acting as the reflected reference light image) with the limit of the effective measurement range, the limit of the detection, or the like, in one of various ways. Also, besides the limit of the effective measurement range and the limit of the detection, the transfinite area may be determined in accordance with the intensity of the reflected reference light representing the presence of the regularly reflected light. Further, in the abnormal light affected area judging device 784, the abnormal light affected area may be determined in accordance with the logical product of the transfinite areas in the manner described above. Alternatively, the abnormal light affected area maybe determined in accordance with the logical sum of the transfinite areas. As another alternative, the abnormal light affected area may be determined in accordance with a specific transfinite area.

Also, in the image composer 790, the incorporation of the abnormal light affected area into the tissue condition image may be performed only when the composed image is to be displayed as a still image. Specifically, the image displaying may be performed such that the abnormal light affected area is displayed only when the tissue condition of the living body tissues 1 is being displayed as a still image on the video monitor 770, and such that the abnormal light affected area is not displayed when the tissue condition of the living body tissues 1 is being displayed as a dynamic image on the video monitor 770. The change-over between the displaying of the still image and the displaying of the dynamic image may be performed by utilizing a hand-operated switch or a foot switch for the operation of the fluorescence endoscope system 900.

Further, as illustrated in FIG. 13, the fluorescence endoscope system 900 may be provided with a displaying change-over switch 791, which acts as the displaying change-over means for manually changing over between an abnormal light affected area displaying mode and an abnormal light affected area non-displaying mode. When the abnormal light affected area is not to be displayed, the displaying change-over switch 791 may be set at the non-displaying mode such that the abnormal light affected area may not be displayed. Specifically, when the displaying change-over switch 791 is set at the non-displaying mode, a non-displaying instruction signal is fed out from the displaying change-over switch 791. The image composer 790 receives the non-displaying instruction signal and ceases the incorporation of the abnormal light affected area into the tissue condition image, and only the tissue condition image is fed out as the composed image from the image composer 790. At this time, the non-displaying instruction signal is also fed into the controller 750. The controller 750 receives the non-displaying instruction signal and controls the image judgment unit 780 such that the image judgment unit 780 may cease the processing for determining the abnormal light affected area. In this manner, the burden to the processing in the image judgment unit 780 is capable of being kept light.

Furthermore, when the tissue condition image and the abnormal light affected area are combined with each other by the image composer 790, the person, who sees the displayed image, may select a displaying form of the abnormal light affected area, such that the abnormal light affected area displayed on the video monitor 770 may be displayed in a desired form (the color, the shape, the pattern, the presence or absence of blinking, and the like).

The judgment in the effective measurement range judging device 781, the effective measurement range judging device 782, the overflow judging device 783, and the abnormal light affected area judging device 784 is not limited to the judgment made in units of a single pixel. For example, the judgment may be made in arbitrary units of nxm pixels desired by the person, who sees the displayed image. As another alternative, with the amount of the operation processing being taken into consideration, the pixels may be thinned out appropriately, and thereafter the comparison may be made. In cases where, for example, the pixels are thinned out, interpolating operations may be performed in accordance with the results of the judgment at neighboring pixels. Also, the judgment may be made with respect to only an area of interest of the person, who sees the displayed image. In such cases, the display color at the areas, for which the judgment is not made, maybe set at a specific color, and the area of interest may thereby be displayed clearly.

Further, in the fluorescence endoscope system 900, in which the second embodiment of the apparatus for displaying a fluorescence image in accordance with the present invention is employed, the ordinary image and the composed image are displayed respectively on the video monitor 760 and the video monitor 770. Alternatively, both the ordinary image and the composed image may be displayed on a single video monitor. In such cases, the change-over between the displaying of the ordinary image and the displaying of the composed image may be performed automatically by being synchronized with the change-over between the displaying of the dynamic image and the displaying of the still image. Alternatively, the person, who sees the displayed image, may make the change-over arbitrarily by utilizing appropriate change-over means.

Furthermore, in the fluorescence endoscope system 900, the GaN type of semiconductor laser 714 and the white light source 711 are provided as two independent devices. Alternatively, by the utilization of an appropriate band-pass filter, a single light source may be utilized as both the excitation light source and the white light source.

Also, in the fluorescence endoscope system 900, the CCD image sensor 707 for the detection of the ordinary image is located at the leading end 700A of the fluorescence endoscope. Alternatively, an image fiber may be utilized to guide the ordinary image from the leading end 700A into an imaging unit, and thereafter the ordinary image may be detected with the CCD image sensor located within the imaging unit. Also, for example, the rotating filter 722 may be altered, and a multi-color mosaic filter may be combined with the image sensor. In this manner, an image fiber and an image sensor may be utilized in common for the detection of the ordinary image, the detection of the fluorescence image, and the detection of the reflected reference light image.

Further, an image sensor combined with a multi-color mosaic filter may be located at the leading end of the fluorescence endoscope. In this manner, a single image sensor may be utilized in common for the detection of the ordinary image, the detection of the fluorescence image, and the detection of the reflected reference light image.

Furthermore, in the fluorescence endoscope system 900, in which the second embodiment of the apparatus for displaying a fluorescence image in accordance with the present invention is employed, the operation processing in the image judgment unit 780 and the operation processing in the tissue condition image forming unit 730 are performed as two independent operations. Alternatively, the operation may be controlled such that, with respect to the abnormal light affected area having been determined in the image judgment unit 780, no operation processing may be performed in the tissue condition image forming unit 730. In such cases, the time required to perform the image processing in the tissue condition image forming unit 730 is capable of being kept short.

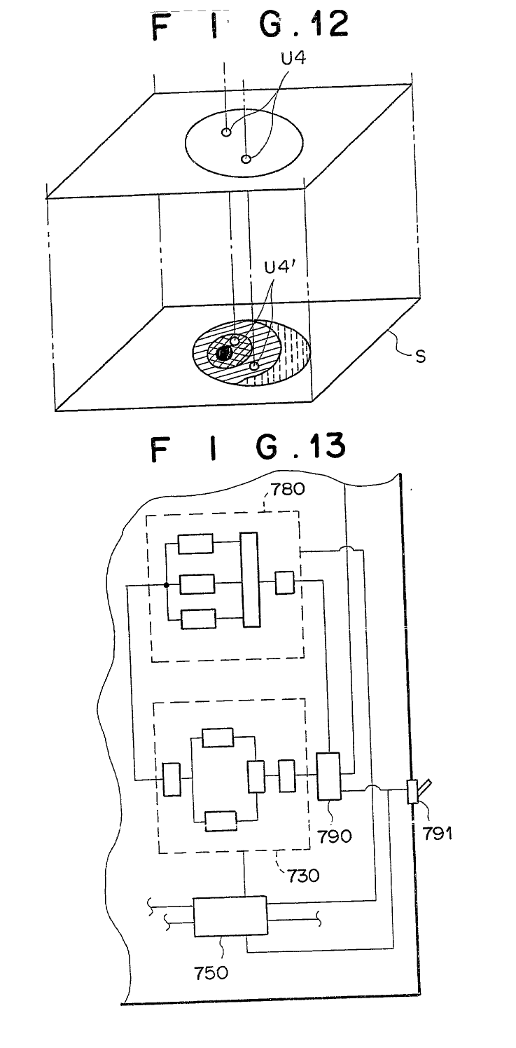

Embodiments of the apparatus for acquiring an endoscope image in accordance with the present invention will be described hereinbelow. FIG. 14 is a schematic view showing a fluorescence endoscope system, in which a first embodiment of the apparatus for acquiring an endoscope image in accordance with the present invention is employed.

With reference to FIG. 14, a fluorescence endoscope system 810, in which the first embodiment of the apparatus for acquiring an endoscope image in accordance with the present invention is employed, comprises a light source unit 110 for radiating out white light Lw and excitation light Le, which has a wavelength of 410 nm. The fluorescence endoscope system 810 also comprises an endoscope unit 210 for receiving the excitation light Le from the light source unit 110, irradiating the excitation light Le via an A-side optical fiber 221a and a B-side optical fiber 221b to the living body tissues 1, detecting images of the living body tissues 1, which images are formed with the excitation light having been reflected by the living body tissues 1 when the excitation light Le is irradiated to the living body tissues 1, with a short-wavelength image sensor 225, and converting the images into electric image signals. The fluorescence endoscope system 810 further comprises a relay unit 310 for receiving the image signals from the endoscope unit 210, performing noise suppression processing, defect compensation processing, image signal processing, and the like, on the received image signals, and converting the image signals into digital two-dimensional image signals. The fluorescence endoscope system 810 still further comprises an operation processing unit 410 for receiving the two-dimensional image signals from the relay unit 310, correcting image signal components contained in the two-dimensional image signals, which image signal components represent regular reflection image areas, in order to obtain a reflection image, and obtaining a fluorescence yield image representing a fluorescence yield. The fluorescence endoscope system 810 also comprises a display device 510 for displaying the fluorescence yield image having been acquired by the operation processing unit 410.

The light source unit 110 is connected to an end face A1 of the A-side optical fiber 221a and an end face B1 of the B-side optical fiber 221b. The white light Lw, which has been produced by a white light source 109, impinges upon a dichroic mirror 111, which reflects light having wavelengths falling within the wavelength region of the excitation light Le and transmits light having wavelengths falling within the wavelength region of the white light Lw.

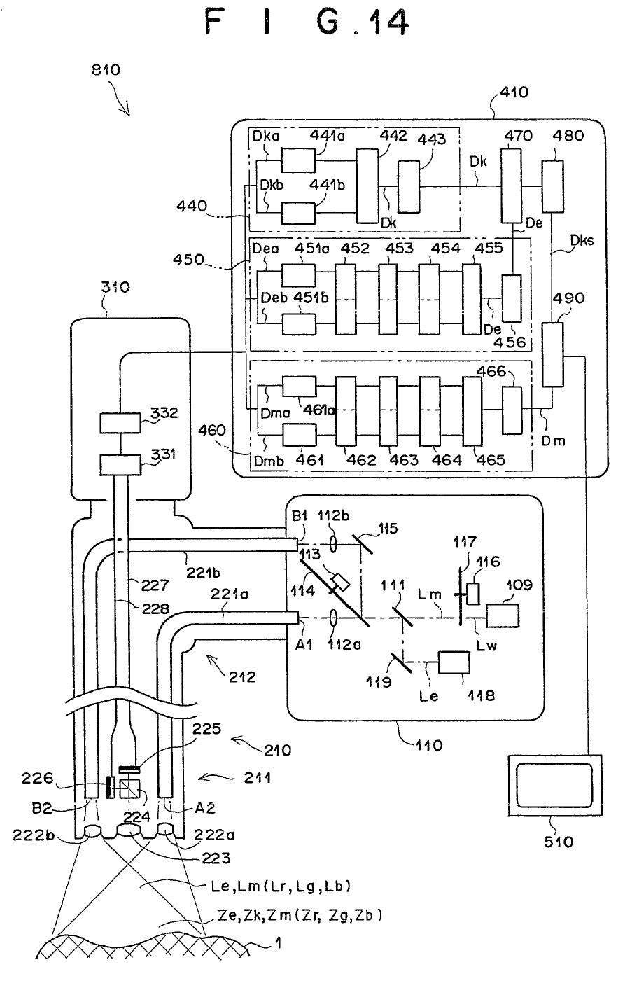

A disk-like rotating filter 117 is located between the white light source 109 and the dichroic mirror 111. As illustrated in FIG. 15, the disk-like rotating filter 117 is provided with R, G, and B filters, i.e. three primary color filters, and a light blocking filter. The disk-like rotating filter 117 is secured for rotation to a rotation shaft of a motor 116. When the disk-like rotating filter 117 is rotated, the white light Lw having been produced by the white light source 109 is divided into red light Lr, green light Lg, and blue light Lb. The group of the red light Lr, the green light Lg, and the blue light Lb constitutes surface sequential light Lm for RGB surface sequential irradiation.

The surface sequential light Lm passes through the dichroic mirror 111 and is split by a rotating reflection-transmission plate 114 so as to follow two optical paths. As illustrated in FIG. 16, the rotating reflection-transmission plate 114 comprises transmission plates 114a, 114a, . . . for transmitting light and reflection plates 114b, 114b, . . . for reflecting light, which are located alternately. The rotating reflection-transmission plate 114 is secured for rotation to a rotation shaft of a motor 113. The surface sequential light Lm, which has passed through the transmission plates 114a, 114a, . . . , is converged by a converging lens 112a and impinges upon the end face A1 of the A-side optical fiber 221a. The surface sequential light Lm, which has been reflected by the reflection plates 114b, 114b, . . . , is reflected by a reflecting mirror 115, is converged by a converging lens 112b, and then impinges upon the end face B1 of the B-side optical fiber 221b.

The excitation light Le, which has been produced by an excitation light source 118, is reflected by a reflecting mirror 119, is then reflected by the dichroic mirror 111, and impinges upon the rotating reflection-transmission plate 114. As in the surface sequential light Lm, the excitation light Le is split by the rotating reflection-transmission plate 114 so as to follow the two optical paths. The split beams of the excitation light Le impinge upon the end face Al of the A-side optical fiber 221a and the end face B1 of the B-sideoptical fiber 221b with different timings.

The endoscope unit 210 comprises a flexible leading end section 211 and a manipulating section 212, which is connected to the light source unit 110 and the relay unit 310. The A-side optical fiber 221a and the B-side optical fiber 221b extend from the manipulating section 212 to the leading end section 211 in the endoscope unit 210.

The A-side optical fiber 221a and an A-side irradiating lens 222a constitute a channel A. The surface sequential light Lm and the excitation light Le, which have impinged upon the end face A1 of the A-side optical fiber 221a, are guided through the A-side optical fiber 221a, radiated out from an end face A2 of the A-side optical fiber 221a, and irradiated via the A-side irradiating lens 222a to the living body tissues 1. Also, the B-side optical fiber 221b and a B-side irradiating lens 222b constitute a channel B. The surface sequential light Lm and the excitation light Le, which have impinged upon the end face B1 of the B-side optical fiber 221b, are guided through the B-side optical fiber 221b, radiated out from an end face B2 of the B-side optical fiber 221b, and irradiated via the B-side irradiating lens 222b to the living body tissues 1.

The image of the living body tissues 1, which is formed with the excitation light having been reflected by the living body tissues 1 when the excitation light Le is irradiated through each of the channel A and the channel B to the living body tissues 1, passes through an objective lens 223 located at the leading end section 211 and impinges upon a dichroic cubic beam splitter 224, which transmits light having a wavelength of 410 nm and reflects light having wavelengths longer than 410 nm. (The image, which is formed with the excitation light having been reflected by the living body tissues 1 when the excitation light Le is irradiated through each of the channel A and the channel B, will hereinbelow referred to as the excitation light image Ze.) The excitation light image Ze passes through the dichroic cubic beam splitter 224, is formed on the short-wavelength image sensor 225, and is converted into an electric image signal. The thus obtained electric image signal is transmitted through a cable 227 to the relay unit 310.

The image of the living body tissues 1, which is formed with the fluorescence produced from the living body tissues 1 when the excitation light Le is irradiated through each of the channel A and the channel B to the living body tissues 1, and the image of the living body tissues 1, which is formed with the surface sequential light having been reflected by the living body tissues 1 when the RGB surface sequential light Lm is irradiated through each of the channel A and the channel B to the living body tissues 1, impinge upon the objective lens 223 with different timings. (The image, which is formed with the fluorescence produced from the living body tissues 1 when the excitation light Le is irradiated through each of the channel A and the channel B, will hereinbelow be referred to as the fluorescence image Zk. Also, the image, which is formed with the surface sequential light having been reflected by the living body tissues 1 when the RGB surface sequential light Lm is irradiated through each of the channel A and the channel B, will hereinbelow be referred to as the surface sequential light image Zm.) Each of the fluorescence image Zk and the surface sequential light image Zm passes through the objective lens 223, and the direction of the optical path of the image is changed by the dichroic cubic beam splitter 224 by an angle of approximately 900. Each of the fluorescence image Zk and the surface sequential light image Zm is then formed on a long-wavelength image sensor 226 and is converted into an electric image signal. The thus obtained electric image signal is transmitted through a cable 228 to the relay unit 310.

The surface sequential light image Zm represents the group of a red light image zr, a green light image Zg, and a blue light image Zb of the living body tissues 1, which images are formed with the light having been reflected by the living body tissues 1 when the red light Lr, and green light Lg, and the blue light Lb acting as the RGB surface sequential light Lm are respectively irradiated to the living body tissues 1.

The relay unit 310 comprises a process circuit section 331 for receiving each of the image signals having been transmitted through the cable 227 and the cable 228, and performing noise suppression processing, defect compensation processing, image signal processing, and the like, on the received image signal. The relay unit 310 also comprises an analog-to-digital converter 332 for converting the image signal into the digital two-dimensional image signal.

The operation processing unit 410 comprises a fluorescence image processing section 440 for performing addition processing on the two-dimensional image signals, which represent the fluorescence images Zk, Zk having been obtained through the channel A and the channel B and have been received from the relay unit 310. The operation processing unit 410 also comprises an excitation light image processing section 450 for performing image processing for removing luminous points due to the regularly reflected light, which are embedded in the excitation light images Ze, Ze having been obtained through the channel A and the channel B and having been converted by the relay unit 310 into the two-dimensional image signals. The operation processing unit 410 further comprises a surface sequential light image processing section 460 for performing image processing for removing luminous points due to the regularly reflected light, which are embedded in the surface sequential light images Zm, Zm having been obtained through the channel A and the channel B and having been converted by the relay unit 310 into the two-dimensional image signals.

The two-dimensional image signals, which have been processed by the fluorescence image processing section 440, and the two-dimensional image signals, which have been processed by the excitation light image processing section 450, are fed into a fluorescence yield calculator 470. In the fluorescence yield calculator 470, operation processing for calculating the fluorescence yield is performed, and a fluorescence yield image signal representing the results of the operation processing is obtained. The fluorescence yield image signal is stored in a fluorescence yield image memory 480. The fluorescence yield image signal, which has been stored in the fluorescence yield image memory 480, and a reflected surface sequential light image signal, which represents a reflection image and has been obtained from the surface sequential light image processing section 460, are fed into a display signal processing circuit 490. In the display signal processing circuit 490, the received two-dimensional image signals are transformed into display signals. The display signals are fed from the display signal processing circuit 490 into the display device 510 and utilized for displaying a visible image.

How the fluorescence endoscope system 810, in which the first embodiment of the apparatus for acquiring an endoscope image in accordance with the present invention is employed, operates will be described hereinbelow.

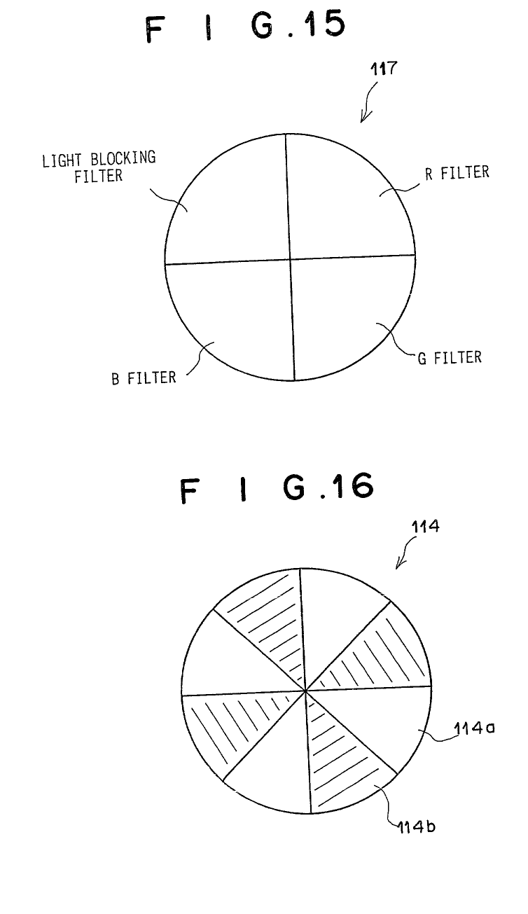

Firstly, timings, with which light irradiation through the channel A and the light irradiation through the channel B are performed, will be described hereinbelow. The white light source 109 is always turned on. The white light Lw having been produced by the white light source 109 passes through the disk-like rotating filter 117. As a result, as illustrated in FIG. 17, the white light Lw is separated successively into the red light Lr, the green light Lg, and the blue light Lb, which act as the surface sequential light Lm. The surface sequential light Lm then passes through the dichroic mirror 111. When the disk-like rotating filter 117 is rotated even further, the white light Lw is blocked by the light blocking filter of the disk-like rotating filter 117. At this stage, one cycle of the rotation of the disk-like rotating filter 117 is completed. When the white light Lw is being blocked by the light blocking filter of the disk-like rotating filter 117, The excitation light source 118 is turned on to produce the excitation light Le. The excitation light Le is reflected by the dichroic mirror 111 and follows the same optical path as the optical path of the red light Lr, the green light Lg, and the blue light Lb and with a timing different from the timings of the red light Lr, the green light Lg, and the blue light Lb.

Each of the red light Lr, the green light Lg, the blue light Lb, and the excitation light Le, which follow the same optical path with the different timings is separated by the rotating reflection-transmission plate 114, which is rotating synchronously with the disk-like rotating filter 117, into beams following the two optical paths. The two beams of each light impinge upon the end face A1 of the channel A and the end face B1 of the channel B with the timings illustrated in FIG. 17. Therefore, a pair of operations, in which the two beams of the light having wavelengths falling within the identical wavelength region are irradiated respectively from the channel A and the channel B toward the living body tissues 1, are iterated, and the wavelength region of the wavelengths of the light beams irradiated to the living body tissues 1 is altered successively.

In the manner described above, the two beams of the light having wavelengths falling within the identical wavelength region are irradiated respectively from the channel A and the channel B toward the living body tissues 1. How the images of the living body tissues 1, which images are formed when the light beams are irradiated to the living body tissues 1, are detected and converted into the two-dimensional image signals and are then subjected to image processing will be described hereinbelow.

When the excitation light Le is irradiated from the channel A to the living body tissues 1, an A-side fluorescence image Zka is formed with the fluorescence produced from the living body tissues 1. The A-side fluorescence image Zka is detected by the long-wavelength image sensor 226 via the objective lens 223 and the dichroic cubic beam splitter 224, and the image signal representing the A-side fluorescence image Zka is obtained from the long-wavelength image sensor 226. The image signal representing the A-side fluorescence image Zka is processed by the relay unit 310 and stored as an A-side fluorescence image signal Dka in an A-side fluorescence image memory 441a of the fluorescence image processing section 440. When the excitation light Le is irradiated from the channel B to the living body tissues 1, a B-side fluorescence image Zkb is formed with the fluorescence produced from the living body tissues 1. A B-side fluorescence image signal Dkb, which represents the B-side fluorescence image Zkb, is obtained in the same manner as that described above and stored in a B-side fluorescence image memory 441b of the fluorescence image processing section 440.

Also, when the excitation light Le is irradiated from the channel A to the living body tissues 1, an A-side excitation light image Zea of the living body tissues 1 is formed with the excitation light reflected by the living body tissues 1. The A-side excitation light image Zea is detected by the short-wavelength image sensor 225 via the objective lens 223 and the dichroic cubic beam splitter 224, and the image signal representing the A-side excitation light image Zea is obtained from the short-wavelength image sensor 225. The image signal representing the A-side excitation light image Zea is processed by the relay unit 310 and stored as an A-side excitation light image signal Dea in an A-side excitation light image memory 451a of the excitation light image processing section 450. When the excitation light Le is irradiated from the channel B to the living body tissues 1, a B-side excitation light image Zeb is formed with the excitation light reflectedby the living body tissues 1. A B-side excitation light image signal Deb, which represents the B-side excitation light image Zeb, is obtained in the same manner as that described above and stored in a B-side excitation light image memory 451b of the excitation light image processing section 450.

When the RGB surface sequential light Lm is irradiated from the channel A to the living body tissues 1, an A-side surface sequential light image Zma of the living body tissues 1 is formed with the surface sequential light reflected by the living body tissues 1. The A-side surface sequential light image Zma is detected by the long-wavelength image sensor 226 via the objective lens 223 and the dichroic cubic beam splitter 224, and the image signal representing the A-side surface sequential light image Zma is obtained from the long-wavelength image sensor 226. The image signal representing the A-side surface sequential light image Zma is processed by the relay unit 310 and stored as an A-side surface sequential light image signal Dma in an A-side surface sequential light image memory 461a of the surface sequential light image processing section 460. (Specifically, an A-side red light image signal Dra, an A-side green light image signal Dga, and an A-side blue light image signal Dba, which act as the A-side surface sequential light image signal Dma, are stored in the A-side surface sequential light image memory 461a of the surface sequential light image processing section 460.) When the RGB surface sequential light Lm is irradiated from the channel B to the living body tissues 1, a B-side surface sequential light image Zmb of the living body tissues 1 is formed with the surface sequential light reflectedby the living body tissues 1. A B-side surface sequential light image signal Dmb, which represents the B-side surface sequential light image Zmb, is obtained in the same manner as that in the A-side surface sequential light image signal Dma and stored in a B-side surface sequential light image memory 461b of the surface sequential light image processing section 460.

In the fluorescence image processing section 440, the A-side fluorescence image signal Dka, which has been stored in the A-side fluorescence image memory 441a, and the B-side fluorescence image signal Dkb, which has been stored in the B-side fluorescence image memory 441b, are fed into an adder 442. In the adder 442, signal values of the A-side fluorescence image signal Dka and the B-side fluorescence image signal Dkb, which signal values represent corresponding pixels in the images represented by the two fluorescence image signals, are added to each other. A fluorescence image signal Dk is obtained from the adder 442. The fluorescence image signal Dk is stored in a fluorescence image memory 443.

In the excitation light image processing section 450, the A-side excitation light image signal Dea, which has been stored in the A-side excitation light image memory 451a, is subjected to low-pass filtering processing, which is performed by a low-pass filter 452, and differentiation filtering processing, which is performed by a differentiation filter 453. The image signal having been obtained from the differentiation filter 453 is then subjected to substitution processing, which is performed by a substitution processor 454. The image signal having been obtained from the substitution processor 454 is fed into an adder 455. Also, the B-side excitation light image signal Deb, which has been stored in the B-side excitation light image memory 451b, is processed in the same manner as that described above and fed into the adder 455. In the adder 455, signal values of the two-dimensional image signals having been obtained through the channel A and the channel B, which signal values represent corresponding pixels in the images represented by the two image signals, are added to each other. A reflected excitation light image signal De is obtained from the adder 455. The reflected excitation light image signal De is stored in an excitation light image memory 456.

In the surface sequential light image processing section 460, in the same manner as that in the excitation light image processing section 450, the A-side surface sequential light image signal Dma, which has been stored in the A-side surface sequential light image memory 461a, is subjected to low-pass filtering processing, which is performed by a low-pass filter 462, and differentiation filtering processing, which is performed by a differentiation filter 463. The image signal having been obtained from the differentiation filter 463 is then subjected to substitution processing, which is performed by a substitution processor 464. The image signal having been obtained from the substitution processor 464 is fed into an adder 465. Also, the B-side surface sequential light image signal Dmb, which has been stored in the B-side surface sequential light image memory 461b, is processed in the same manner as that described above and fed into the adder 465. In the adder 465, signal values of the two-dimensional image signals having been obtained through the channel A and the channel B, which signal values represent corresponding pixels in the images represented by the two image signals, are added to each other. A reflected surface sequential light image signal Dm is obtained from the adder 465. The reflected surface sequential light image signal Dm is stored in a surface sequential light image memory 466.

The fluorescence image signal Dk, which has been stored in the fluorescence image memory 443, and the reflected excitation light image signal De, which has been stored in the excitation light image memory 456, are fed into the fluorescence yield calculator 470. In the fluorescence yield calculator 470, a fluorescence yield image signal Dks representing the fluorescence yield is calculated. Specifically, the division represented by the formula shown below is performed with respect to each of the pixels in the images represented by the fluorescence image signal Dk and the reflected excitation light image signal De, and the values of the fluorescence yield image signal Dks are calculated.

Dks=Dk/De

The fluorescence yield image signal Dks is stored in the fluorescence yield image memory 480.

Thereafter, the reflected surface sequential light image signal Dm, which has been stored in the surface sequential light image memory 466, and the fluorescence yield image signal Dks, which has been stored in the fluorescence yield image memory 480, are fed into the display signal processing circuit 490. In the display signal processing circuit 490, the two-dimensional image signals are transformed into display signals. The display signals are fed into the display device 510 and utilized for simultaneously displaying a reflected surface sequential light image and a fluorescence yield image.

How the processing is performed in the excitation light image processing section 450 will hereinbelow be described in detail. As illustrated in FIG. 18, the image, which is represented by the A-side excitation light image signal Dea having been stored in the A-side excitation light image memory 451a, is constituted of a large luminous point Pa1, a small luminous point Pa2, and areas representing the shape of the living body tissues 1. The large luminous point Pa1 and the small luminous point Pa2 occur due to the detection of the excitation light, which was regularly reflected by the living body tissues 1 when the excitation light Le was irradiated from the channel A to the living body tissues 1, by the short-wavelength image sensor 225. The areas representing the shape of the living body tissues 1 are formed with the excitation light, which was reflected through scattering reflection by the living body tissues 1 when the excitation light Le was irradiated from the channel A to the living body tissues 1, and which was detected the short-wavelength image sensor 225.

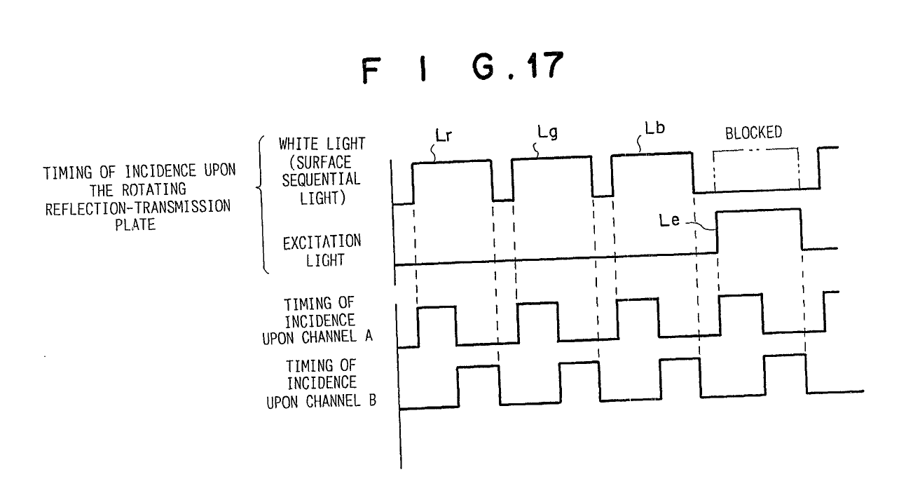

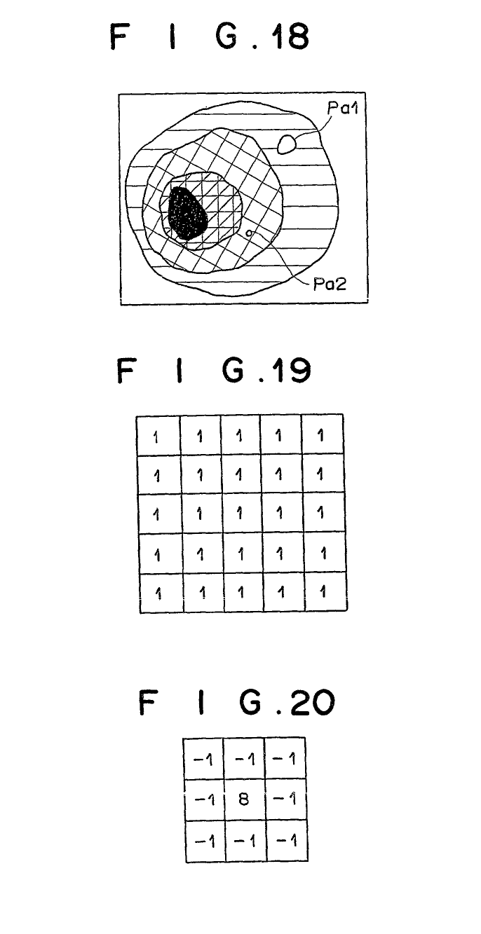

The low-pass filter 452 performs the low-pass filtering processing on the two-dimensional image signal representing the image, in which the large luminous point Pa1and the small luminous point Pa2 are embedded. Specifically, as illustrated in FIG. 19, the low-pass filtering processing is performed on the two-dimensional image signal and with a 5×5 moving average operator. With the low-pass filtering processing, the signal values representing the area of the small luminous point Pa2 become identical with the signal values representing the surrounding areas, which have been formed with the scattering reflection, and the area of the small luminous point Pa2 is removed. However, with the low-pass filtering processing, the large luminous point Pa1 constituted of low frequency components cannot be removed. Therefore, in the differentiation filter 453, sharp rising at the boundary of the large luminous point Pa1 is detected, and the area of the large luminous point Pa1 is specified. Specifically, as illustrated in FIG. 20, the differentiation processing with a 3×3 differentiation operator is performed on the two-dimensional image signal representing the image, in which the large luminous point Pa1 is embedded. With the differentiation processing, the area exceeding a predetermined threshold value is specified as the regular reflection image area. Also, a signal, which represents the position of the specified regular reflection image area, and the two-dimensional image signal, which has been obtained from the low-pass filtering processing, are fed into the substitution processor 454. In the substitution processor 454, as illustrated in FIG. 21, the image values of the specified regular reflection image area representing the large luminous point Pa1 are substituted by a mean value of image values of an area Qa1 surrounding the regular reflection image area.

Also, the B-side excitation light image signal Deb, which has been stored in the B-side excitation light image memory 451b, represents an image approximately identical with the image represented by the A-side excitation light image signal Dea. In the same manner as that in the A-side excitation light image signal Dea, the B-side excitation light image signal Deb is subjected to the low-pass filtering processing, the differentiation processing, and the substitution processing for removing luminous points due to the regularly reflected light.

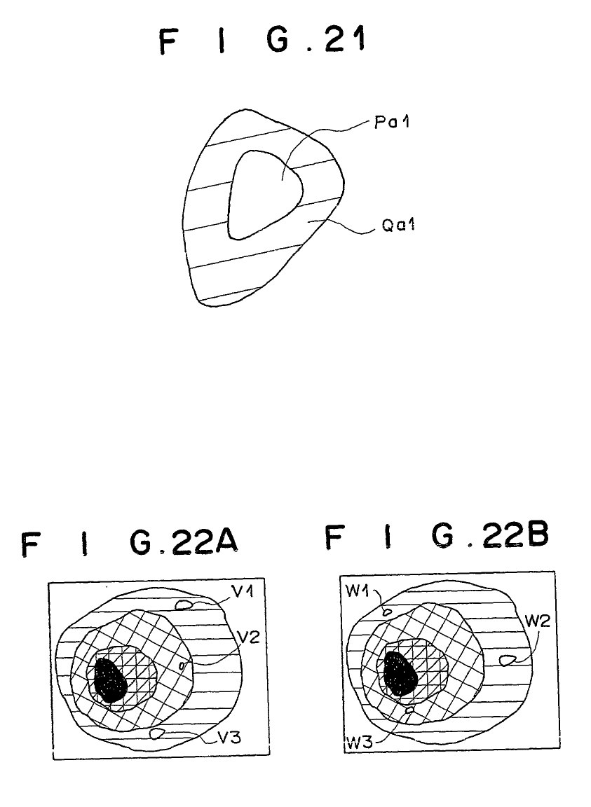

In both the cases where the light is irradiated from the channel A and the cases where the light is irradiated from the channel B, the image of the living body tissues 1 is guided through the common optical path of the optical system for detecting the image. Therefore, as illustrated in FIG. 22A and FIG. 22B, the image formed with the excitation light, which has been reflected through the scattering reflection from the living body tissues 1 when the excitation light Le is irradiated from the channel A to the living body tissues 1, and the image formed with the excitation light, which has been reflected through the scattering reflection from the living body tissues 1 when the excitation light Le is irradiated from the channel B to the living body tissues 1, coincide with each other. In the two images illustrated in FIG. 22A and FIG. 22B, only the positions of luminous points v1, v2, v3, which are formed due to the regularly reflected light occurring when the excitation light Le is irradiated from the channel A to the living body tissues 1, and the positions of luminous points w1, w2, w3, which are formed due to the regularly reflected light occurring when the excitation light Le is irradiated from the channel B to the living body tissues 1, vary for the two images. The two kinds of the two-dimensional image signals representing the images obtained by light irradiation from the channel A and the channel B, which image signals have been obtained from the substitution processing described above, are fed into the adder 455. In the adder 455, the two kinds of the two-dimensional image signals are added to each other and averaged. In this manner, the adverse effects of the luminous points occurring due to the regularly reflected light are reduced even further. A two-dimensional image signal, which has been obtained from the adder 455 and in which the adverse effects of the luminous points have been reduced, is fed as a reflected excitation light image signal Dr into the excitation light image memory 456 and stored in the excitation light image memory 456.

In the surface sequential light image processing section 460, the image processing is performed in the same manner as that in the excitation light image processing section 450. The A-side surface sequential light image signal Dma and the B-side surface sequential light image signal Dmb, which have been fed into the surface sequential light image processing section 460, are subjected to the low-pass filtering processing, the differentiation processing, the substitution processing, and the addition processing for removing the luminous points. A two-dimensional image signal, which has been obtained from the addition processing, is fed as a reflected surface sequential light image signal Dm into the surface sequential light image memory 466 and stored in the surface sequential light image memory 466.

The processing with respect to the regular reflection image areas is performed in the manner described above. Therefore, the problems are capable of being prevented from occurring in that the seeing of the state of the living body tissues 1 on the reflected surface sequential light image (i.e., the reflection image) and the fluorescence yield image, which are displayed ultimately, is obstructed by the adverse effects of the luminous points occurring due to the regularly reflected light. Accordingly, the tissue condition of the living body tissues 1 is capable of being discriminated accurately.

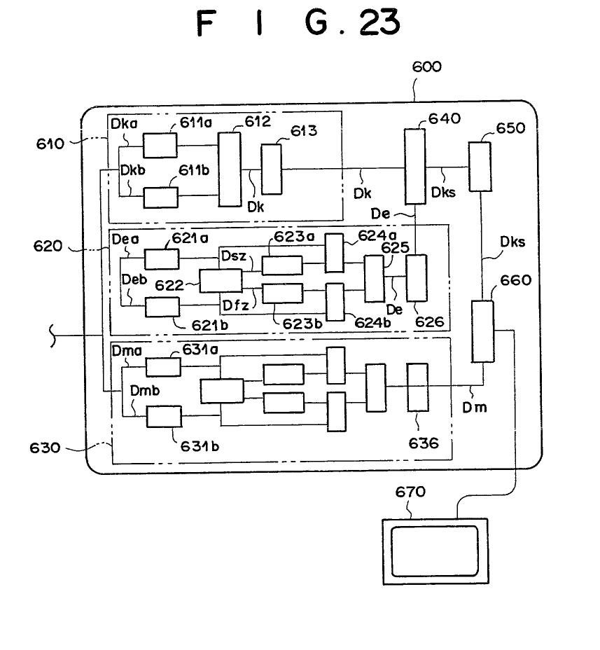

A second embodiment of the apparatus for acquiring an endoscope image in accordance with the present invention will be described hereinbelow. FIG. 23 is a block diagram showing an operation processing unit employed in a fluorescence endoscope system, in which the second embodiment of the apparatus for acquiring an endoscope image in accordance with the present invention is employed. The second embodiment of the apparatus for acquiring an endoscope image in accordance with the present invention is basically identical with the first embodiment of the apparatus for acquiring an endoscope image in accordance with the present invention, except that an operation processing unit 600 is employed in lieu of the operation processing unit 410 shown in FIG. 14. As illustrated in FIG. 23, the operation processing unit 600 comprises a fluorescence image processing section 610, an excitation light image processing section 620, a surface sequential light image processing section 630, a fluorescence yield calculator 640, a fluorescence yield image memory 650, and a display signal processing circuit 660.

As in the first embodiment of the apparatus for acquiring an endoscope image in accordance with the present invention, the A-side fluorescence image Zka and the B-side fluorescence image Zkb of the living body tissues 1 are formed respectively with the fluorescence, which has been produced from the living body tissues 1 when the excitation light Le is irradiated from the channel A to the living body tissues 1, and the fluorescence, which has been produced from the living body tissues 1 when the excitation light Le is irradiated from the channel B to the living body tissues 1. Also, the A-side excitation light image Zea and the B-side excitation light image Zeb of the living body tissues 1 are formed respectively with the reflected excitation light, which has been reflected by the living body tissues 1 when the excitation light Le is irradiated from the channel A to the living body tissues 1, and the reflected excitation light, which has been reflected by the living body tissues 1 when the excitation light Le is irradiated from the channel B to the living body tissues 1. Further, the A-side surface sequential light image Zma and the B-side surface sequential light image Zmb of the living body tissues 1 are formed respectively with the reflected surface sequential light, which has been reflected by the living body tissues 1 when the surface sequential light Lm is irradiated from the channel A to the living body tissues 1, and the reflected surface sequential light, which has been reflected by the living body tissues 1 when the surface sequential light Lm is irradiated from the channel B to the living body tissues 1. The thus formed images are detected, converted into the image signals, subjected to the analog-to-digital conversion, and fed as the two-dimensional image signals into the operation processing unit 600. In the operation processing unit 600, the A-side fluorescence image signal Dka and the B-side fluorescence image signal Dk bare stored respectively in an A-side fluorescence image memory 611a and a B-side fluorescence image memory 611b of the fluorescence image processing section 610. Also, the A-side excitation light image signal Dea and the B-side excitation light image signal Deb are stored respectively in an A-side excitation light image memory 621a and a B-side excitation light image memory 621b of the excitation light image processing section 620. Further, the A-side surface sequential light image signal Dma and the B-side surface sequential light image signal Dmb are stored respectively in an A-side surface sequential light image memory 631a and a B-side surface sequential light image memory 631b of the surface sequential light image processing section 630.

In the fluorescence image processing section 610, the A-side fluorescence image signal Dka, which has been stored in the A-side fluorescence image memory 611a, and the B-side fluorescence image signal Dkb, which has been stored in the B-side fluorescence image memory 611b, are fed into an adder 612. In the adder 612, the signal values of the A-side fluorescence image signal Dka and the B-side fluorescence image signal Dkb, which signal values represent corresponding pixels in the images represented by the two fluorescence image signals, are added to each other. The fluorescence image signal Dk is obtained from the adder 612. The fluorescence image signal Dk is stored in a fluorescence image memory 613.

In the excitation light image processing section 620, the A-side excitation light image signal Dea, which has been stored in the A-side excitation light image memory 621a, and the B-side excitation light image signal Deb, which has been stored in the B-side excitation light image memory 621b, are fed into a subtraction device 622. In the subtraction device 622, the B-side excitation light image signal Deb is subtracted from the A-side excitation light image signal Dea. Also, in the subtraction device 622, image values of a two-dimensional image signal, which has been obtained from the subtraction, is compared with a positive threshold value Ga and a negative threshold value Gb, which have been stored previously in the subtraction device 622. An area, which is associated with the image values larger than the positive threshold value Ga, is stored as a regular reflection image area signal Dsz in a regular reflection image area memory 623a. Also, an area, which is associated with the image values smaller than the negative threshold value Gb, is stored as a regular reflection image area signal Dfz in a regular reflection image area memory 623b.

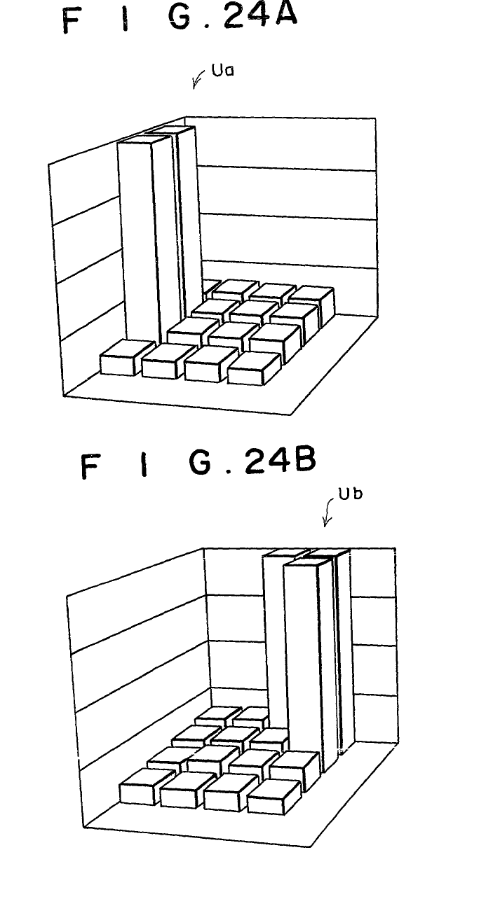

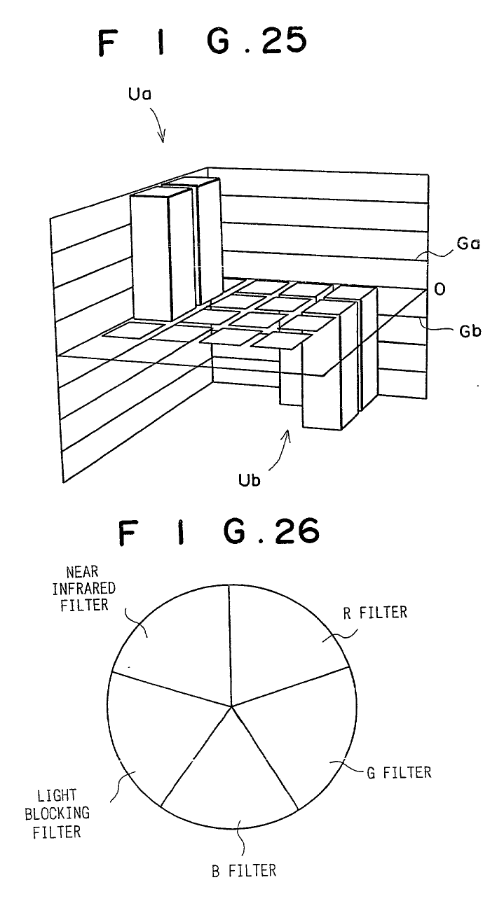

Specifically, as illustrated in FIG. 24A, image values of an area Ua corresponding to a luminous point occurring due to the regularly reflected light, which image values are contained in the A-side excitation light image signal Dea, take values markedly larger than the image values of the other areas, which areas are formed with only the diffuse reflection light. Also, as illustrated in FIG. 24B, image values of an area Ub corresponding to a luminous point occurring due to the regularly reflected light, which image values are contained in the B-side excitation light image signal Deb, take values markedly larger than the image values of the other areas, which areas are formed with only the diffuse reflection light. As illustrated in FIG. 25, when the values of the B-side excitation light image signal Deb are subtracted from the values of the A-side excitation light image signal Dea, the image values of the area Ua take markedly large positive values, and the image values of the area Ub take markedly small negative values. Also, as for the other areas, which are formed with only the diffuse reflection light, little difference occurs between the image values, which are obtained through the irradiation of the excitation light Le from the channel A, and the image values, which are obtained through the irradiation of the excitation light Le from the channel B. Therefore, as illustrated in FIG. 25, the image values of the other areas take values close to 0. Accordingly, the image values of the area Ua of the luminous point, which is formed with the excitation light irradiated from the channel A, and the image values of the area Ub of the luminous point, which is formed with the excitation light irradiated from the channel B, take values outside the range sandwiched between the positive threshold value Ga and the negative threshold value Gb. As a result, the areas Ua and Ub are specified as the regular reflection image areas. The area Ua of the luminous point, which is formed with the excitation light irradiated from the channel A, is stored as the regular reflection image area signal Dsz in the regular reflection image are a memory 623a. Also, the area Ub of the luminous point, which is formed with the excitation light irradiated from the channel B, is stored as the regular reflection image area signal Dfz in the regular reflection image area memory 623b.

Thereafter, the regular reflection image area signal Dsz, which has been stored in the regular reflection image area memory 623a, and the A-side excitation light image signal Dea, which has been stored in the A-side excitation light image memory 621a, are fed into a substitution processor 624a. In the substitution processor 624a, the image values of the area, which has been specified as the regular reflection image area, are substituted by a mean value of the image values of the surrounding area. A two-dimensional image signal, which has been obtained from the substitution processor 624a, is fed into an adder 625. Also, the regular reflection image area signal Dfz, which has been stored in the regular reflection image area memory 623b, and the B-side excitation light image signal Deb, which has been stored in the B-side excitation light image memory 621b, are fed into a substitution processor 624b. In the substitution processor 624b, the image values of the area, which has been specified as the regular reflection image area, are substituted by a mean value of the image values of the surrounding area. A two-dimensional image signal, which has been obtained from the substitution processor 624b, is fed into the adder 625. In the adder 625, the two received two-dimensional image signals are added to each other and averaged. A two-dimensional image signal, in which the adverse effects of the luminous points have been reduced even further, is obtained from the adder 625. The two-dimensional image signal obtained from the adder 625 is fed as the reflected excitation light image signal De into an excitation light image memory 626 and stored in the excitation light image memory 626.

The reflected excitation light image signal De, which has been stored in the excitation light image memory 626, and the fluorescence image signal Dk, which has been stored in the fluorescence image memory 613, are fed into the fluorescence yield calculator 640. In the fluorescence yield calculator 640, the fluorescence yield image signal Dks representing the fluorescence yield is calculated. Specifically, the division represented by the formula shown below is performed.

Dks=Dk/De

The fluorescence yield image signal Dks is stored in the fluorescence yield image memory 650.

In the surface sequential light image processing section 630, in the same manner as that in the excitation light image processing section 620, a two-dimensional image signal, which has been processed for removing the luminous points due to the surface sequential light, is obtained. The thus obtained two-dimensional image signal is stored as the reflected surface sequential light image signal Dm in a surface sequential light image memory 636.

The reflected surface sequential light image signal Dm, which has been stored in the surface sequential light image memory 636, and the fluorescence yield image signal Dks, which has been stored in the fluorescence yield image memory 650, are fed into the display signal processing circuit 660. In the display signal processing circuit 660, the two-dimensional image signals are transformed into display signals. The display signals are fed into a display device 670 and utilized for simultaneously displaying a reflection image and a fluorescence yield image.

The substitution processing described above is not limited to the processing, in which the image values of the regular reflection image area are substituted by the mean value of the image values of the surrounding area. The substitution processing may be altered to, for example, processing for calculating new image values of the regular reflection image area by extrapolating or interpolating operations utilizing the image values of the surrounding area.

Also, the processing with the low-pass filter and the processing with the differentiation filter may be one-dimensional filtering processing.

Further, the operator of the low-pass filter is not limited to the moving average operator and may be a Gaussian mean operator, or the like.

Furthermore, the technique for calculating the fluorescence yield is not limited to the technique, in which the fluorescence yield is calculated from the two-dimensional image signal obtained from the fluorescence image Zk and the two-dimensional image signal obtained from the excitation light image Ze. For example, in lieu of the excitation light image Ze, the red light image Zr, which is one of the R, G, and B surface sequential light images Zm, may be utilized in order to obtain the fluorescence yield. Alternatively, the fluorescence yield may be obtained by utilizing the two-dimensional image signal corresponding to the luminance signal among the video signals, which is calculated from addition and subtraction performed on the R, G, and B surface sequential light images Zm. As another alternative, the fluorescence yield may be obtained in accordance with a near infrared light image, which is obtained through the irradiation of near infrared light. In order for the near infrared light image to be obtained, a rotating filter illustrated in FIG. 26 may be utilized. The rotating filter illustrated in FIG. 26 is formed by adding a near infrared filter, which transmits only the light having wavelengths falling within the near infrared wavelength region, to the rotating filter for separating the white light into the R, G, and B three primary color light beams. With the rotating filter illustrated in FIG. 26, the white light may be separated into the red light, the green light, the blue light, and the near infrared light. The near infrared light may be irradiated to the living body tissues 1, and the two-dimensional image signal in accordance with the near infrared light image may thereby be obtained.

As each of the short-wavelength image sensor 225 and the long-wavelength image sensor 226, a back-incidence type of charge coupled device (CCD) image sensor, which has a high quantum efficiency and a high sensitivity with respect to the short wavelength region of visible light, should preferably be employed. In such cases, the fluorescence image Zk, the surface sequential light image Zm, and the excitation light image Ze are capable of being detected with one image sensor. In such cases, since the light intensity of the fluorescence image Zk is very low, it is necessary that the intensity of the excitation light Le irradiated to the living body tissues 1, the intensity of the surface sequential light Lm irradiated to the living body tissues 1, the transmittance of each filter, the exposure time for the image detection, and the like, be adjusted appropriately in accordance with the characteristics, such as the light receiving sensitivity and the dynamic range, of the back-incidence type of CCD image sensor.

What is claimed is:

1. A method of displaying a fluorescence image, wherein operation processing is performed on a first fluorescence image having been obtained by detecting fluorescence components of fluorescence having been produced from living body tissues exposed to excitation light, which fluorescence components have wavelengths falling within a specific wavelength region, and at least either one of a second fluorescence image having been obtained by detecting fluorescence components of the fluorescence, which fluorescence components have wavelengths falling within a wavelength region different from the specific wavelength region, and a reflected reference light image having been obtained by detecting reflected reference light, which has been reflected from the living body tissues when reference light is irradiated to the living body tissues, a tissue condition image, which represents a tissue condition of the living body tissues and which has been compensated for a distance to the living body tissues, is formed with the operation processing, and the thus formed tissue condition image is displayed, the method comprising the steps of:
i) making a judgment as to whether each of image areas embedded in the tissue condition image is an abnormal light affected area, which has been affected by light having an intensity equal to at least a specified value, or a normal light detection area, which has been formed with light having an intensity lower than the specified value, the judgment being made in accordance with at least one image, which is among the first fluorescence image, the second fluorescence image, and the reflected reference light image, and
ii) displaying the abnormal light affected area in a form different from the normal light detection area.

2. An apparatus for displaying a fluorescence image, wherein operation processing is performed on a first fluorescence image having been obtained by detecting fluorescence components of fluorescence having been produced from living body tissues exposed to excitation light, which fluorescence components have wavelengths falling within a specific wavelength region, and at least either one of a second fluorescence image having been obtained by detecting fluorescence components of the fluorescence, which fluorescence components have wavelengths falling within a wavelength region different from the specific wavelength region, and a reflected reference light image having been obtained by detecting reflected reference light, which has been reflected from the living body tissues when reference light is irradiated to the living body tissues, a tissue condition image, which represents a tissue condition of the living body tissues and which has been compensated for a distance to the living body tissues, is formed with the operation processing, and the thus formed tissue condition image is displayed, the apparatus comprising:
i) judgment means for making a judgment as to whether each of image areas embedded in the tissue condition image is an abnormal light affected area, which has been affected by light having an intensity equal to at least a specified value, or a normal light detection area, which has been formed with light having an intensity lower than the specified value, the judgment being made in accordance with at least one image, which is among the first fluorescence image, the second fluorescence image, and the reflected reference light image, and
ii) abnormal light affected area displaying means for receiving an output from the judgment means and displaying the abnormal light affected area in a form different from the normal light detection area in accordance with the output received from the judgment means.

3. An apparatus for displaying a fluorescence image as defined in claim 2 wherein the specified value is determined in accordance with an intensity of the reflected reference light, which intensity indicates the presence of regularly reflected light, in the reflected reference light image.

4. An apparatus for displaying a fluorescence image as defined in claim 2 wherein the specified value is determined in accordance with a limit of the detection in at least one image, which is among the first fluorescence image, the second fluorescence image, and the reflected reference light image.

5. An apparatus for displaying a fluorescence image as defined in claim 2 wherein the specified value is determined in accordance with a limit of an effective measurement range in at least one image, which is among the first fluorescence image, the second fluorescence image, and the reflected reference light image.

6. An apparatus for displaying a fluorescence image as defined in claim 2, 3, 4, or 5 where in the abnormal light affected area displaying means displays the abnormal light affected area in the form different from the normal light detection area only in cases where the tissue condition image is displayed as a still image.

7. An apparatus for displaying a fluorescence image as defined in claim 2, 3, 4, or 5 wherein the tissue condition image represents a fluorescence yield.

8. An apparatus for displaying a fluorescence image as defined in claim 2, 3, 4, or 5 wherein the tissue condition image represents a normalized fluorescence intensity.

9. An apparatus for displaying a fluorescence image as defined in claim 4 wherein at least one image, which is among the first fluorescence image, the second fluorescence image, and the reflected reference light image, is obtained from photoelectric detection of light with an image sensor, and the limit of the detection corresponds to a saturation value of an output of the image sensor.

10. An apparatus for displaying a fluorescence image as defined in claim 5 wherein a calculation is made to find a mean value of detected values of at least either one of the first fluorescence image and the second fluorescence image, which have been obtained by detecting the fluorescence having been produced from normal tissues when the excitation light is irradiated to the normal tissues spaced apart by a predetermined distance from an excitation light radiating-out point, and the specified value in accordance with the limit of the effective measurement range is determined in accordance with a value, which is obtained by adding a value representing a variation of the detected values to the thus calculated mean value.

11. An apparatus for displaying a fluorescence image as defined in claim 2, 3, 4, 5, 9, or 10 wherein the abnormal light affected area displaying means displays the abnormal light affected area as a color area in cases where the normal light detection area is displayed as a monochromatic area, and the abnormal light affected area displaying means displays the abnormal light affected area as a monochromatic area in cases where the normal light detection area is displayed as a color area.

12. An apparatus for displaying a fluorescence image as defined in claim 2, 3, 4, 5, 9, or 10 wherein the abnormal light affected area displaying means displays the abnormal light affected area as a blinking area.

13. An apparatus for displaying a fluorescence image as defined in claim 2, 3, 4, 5, 9, or 10 wherein the apparatus further comprises displaying change-over means for manually changing over between an abnormal light affected area displaying mode and an abnormal light affected area non-displaying mode.

14. An apparatus for displaying a fluorescence image as defined in claim 2, 3, 4, 5, 9, or 10 wherein the apparatus is constituted as an endoscope system provided with an endoscope tube to be inserted into a living body.

15. An apparatus for displaying a fluorescence image as defined in claim 2, 3, 4, 5, 9, or 10 wherein the apparatus further comprises a light source for producing the excitation light, and the light source is a GaN type of semiconductor laser.

16. A method of acquiring an endoscope image, comprising the steps of:
i) irradiating light to living body tissues,
ii) detecting reflected light, which has been reflected from the living body tissues when the light is irradiated to the living body tissues, as an image, and
iii) acquiring a reflection image from the image, which has been obtained by detecting the reflected light,
wherein the reflection image is acquired by performing low-pass filtering processing on the image, which has been obtained from the detection of the reflected light.

17. A method of acquiring an endoscope image, comprising the steps of:
i) irradiating light to living body tissues,
ii) detecting reflected light, which has been reflected from the living body tissues when the light is irradiated to the living body tissues, as an image, and
iii) acquiring a reflection image from the image, which has been obtained by detecting the reflected light,
wherein the reflection image is acquired by:
performing differentiation filtering processing on the image, which has been obtained from the detection of the reflected light, in order to specify a regular reflection image area, which is embedded in the image having been obtained from the detection of the reflected light and is affected by regularly reflected light of the light having been irradiated to the living body tissues, and
substituting an image value within the regular reflection image area by a corrected value, which is determined in accordance with image values at an area surrounding the regular reflection image area.

18. A method of acquiring an endoscope image, comprising the steps of:
i) irradiating light to living body tissues,
ii) detecting reflected light, which has been reflected from the living body tissues when the light is irradiated to the living body tissues, as an image, and
iii) acquiring a reflection image from the image, which has been obtained by detecting the reflected light,
wherein the irradiation of the light is performed from two different positions and with different timings,
the reflected light, which has been reflected from the living body tissues when the light is irradiated from one of the two different positions to the living body tissues, and the reflected light, which has been reflected from the living body tissues when the light is irradiated from the other position to the living body tissues, are detected respectively as two images, and
the reflection image is acquired by:
calculating a difference between the two detected images in order to specify regular reflection image areas, which are embedded respectively in the two detected images and are affected by regularly reflected light of the light having been irradiated to the living body tissues,
substituting an image value within each of the regular reflection image areas, which are embedded respectively in the two detected images, by a corrected value, which is determined in accordance with image values at an area surrounding the corresponding regular reflection image area, and
adding two images, which have been obtained from the substitution, to each other.

19. A method of acquiring an endoscope image, comprising the steps of:
i) irradiating light to living body tissues,
ii) detecting reflected light, which has been reflected from the living body tissues when the light is irradiated to the living body tissues, as an image, and
iii) acquiring a reflection image from the image, which has been obtained by detecting the reflected light,
wherein the irradiation of the light is performed from two different positions and with different timings,
the reflected light, which has been reflected from the living body tissues when the light is irradiated from one of the two different positions to the living body tissues, and the reflected light, which has been reflected from the living body tissues when the light is irradiated from the other position to the living body tissues, are detected respectively as two images, and
the reflection image is acquired by:
performing low-pass filtering processing on each of the two detected images, and
adding two images, which have been obtained from the low-pass filtering processing, to each other.

20. An apparatus for acquiring an endoscope image, comprising:
i) irradiation means for irradiating light to living body tissues,
ii) detection means for detecting reflected light, which has been reflected from the living body tissues when the light is irradiated to the living body tissues, as an image, and
iii) image acquiring means for acquiring a reflection image from the image, which has been obtained by detecting the reflected light,
wherein the image acquiring means acquires the reflection image by performing low-pass filtering processing on the image, which has been obtained from the detection of the reflected light.

21. An apparatus for acquiring an endoscope image as defined in claim 20 wherein the low-pass filtering processing is one-dimensional low-pass filtering processing.

22. An apparatus for acquiring an endoscope image as defined in claim 20 wherein the low-pass filtering processing is two-dimensional low-pass filtering processing.

23. An apparatus for acquiring an endoscope image, comprising:
i) irradiation means for irradiating light to living body tissues,
ii) detection means for detecting reflected light, which has been reflected from the living body tissues when the light is irradiated to the living body tissues, as an image, and
iii) image acquiring means for acquiring a reflection image from the image, which has been obtained by detecting the reflected light,
wherein the image acquiring means acquires the reflection image by:
performing differentiation filtering processing on the image, which has been obtained from the detection of the reflected light, in order to specify a regular reflection image area, which is embedded in the image having been obtained from the detection of the reflected light and is affected by regularly reflected light of the light having been irradiated to the living body tissues, and
substituting an image value within the regular reflection image area by a corrected value, which is determined in accordance with image values at an area surrounding the regular reflection image area.

24. An apparatus for acquiring an endoscope image as defined in claim 23 wherein the differentiation filtering processing is one-dimensional differentiation filtering processing.

25. An apparatus for acquiring an endoscope image as defined in claim 23 wherein the differentiation filtering processing is two-dimensional differentiation filtering processing.

26. An apparatus for acquiring an endoscope image, comprising:
i) irradiation means for irradiating light to living body tissues,
ii) detection means for detecting reflected light, which has been reflected from the living body tissues when the light is irradiated to the living body tissues, as an image, and
iii) image acquiring means for acquiring a reflection image from the image, which has been obtained by detecting the reflected light,
wherein the irradiation means irradiates the light from two different positions and with different timings to the living body tissues,
the detection means detects the reflected light, which has been reflected from the living body tissues when the light is irradiated from one of the two different positions to the living body tissues, and the reflected light, which has been reflected from the living body tissues when the light is irradiated from the other position to the living body tissues, respectively as two images, and
the image acquiring means acquires the reflection image by:
calculating a difference between the two detected images in order to specify regular reflection image areas, which are embedded respectively in the two detected images and are affected by regularly reflected light of the light having been irradiated to the living body tissues,
substituting an image value within each of the regular reflection image areas, which are embedded respectively in the two detected images, by a corrected value, which is determined in accordance with image values at an area surrounding the corresponding regular reflection image area, and
adding two images, which have been obtained from the substitution, to each other.

27. An apparatus for acquiring an endoscope image, comprising:
i) irradiation means for irradiating light to living body tissues,
ii) detection means for detecting reflected light, which has been reflected from the living body tissues when the light is irradiated to the living body tissues, as an image, and
iii) image acquiring means for acquiring a reflection image from the image, which has been obtained by detecting the reflected light,
wherein the irradiation means irradiates the light from two different positions and with different timings to the living body tissues,
the detection means detects the reflected light, which has been reflected from the living body tissues when the light is irradiated from one of the two different positions to the living body tissues, and the reflected light, which has been reflected from the living body tissues when the light is irradiated from the other position to the living body tissues, respectively as two images, and
the image acquiring means acquires the reflection image by:
performing low-pass filtering processing on each of the two detected images, and
adding two images, which have been obtained from the low-pass filtering processing, to each other.

28. An apparatus for acquiring an endoscope image as defined in claim 20, 21, 22, 23, 24, 25, 26, or 27 wherein the apparatus further comprises excitation light irradiating means for irradiating excitation light to the living body tissues, the excitation light causing the living body tissues to produce fluorescence, and fluorescence image detecting means for detecting the fluorescence, which has been produced from the living body tissues when the excitation light is irradiated to the living body tissues, as a fluorescence image, and
the image acquiring means acquires a fluorescence yield image in accordance with a ratio of the fluorescence image to the reflection image.

29. An apparatus for acquiring an endoscope image as defined in claim 28 wherein the reflection image is an image formed with reflected light of the excitation light.

30. An apparatus for acquiring an endoscope image as defined in claim 28 wherein the reflection image is an image formed with reflected light of near infrared light, which has been irradiated by the irradiation means to the living body tissues.

31. An apparatus for acquiring an endoscope image as defined in claim 28 wherein the reflection image is an image formed with reflected light of light, which has wavelengths falling within a red wavelength region and has been irradiated by the irradiation means to the living body tissues.

32. An apparatus for acquiring an endoscope image as defined in claim 28 wherein the reflection image is an image formed with a luminance signal having been formed in accordance with the reflected light of the light, which has been irradiated by the irradiation means to the living body tissues.
